(19) United States
(12) Patent Application Publication
(10) Pub. No.: US 2021/0220210 A1
(43) Pub. Date: Jul. 22, 2021

(54) APPARATUS AND METHODS TO TREAT HEADACHES

(71) Applicant: Equinox Ophthalmic, Inc., Sioux Falls, SD (US)

(72) Inventors: John Berdahl, Sioux Falls, SD (US); Paul Byoungjae Yoo, Aliso Viejo, CA (US); Matthew Donald Larson, Newport Beach, CA (US); Gary Berman, Laguna Beach, CA (US); George Tsai, Mission Viejo, CA (US)

(21) Appl. No.: 17/225,539

(22) Filed: Apr. 8, 2021

Related U.S. Application Data
(63) Continuation of application No. PCT/US2019/055515, filed on Oct. 10, 2019.
(60) Provisional application No. 62/743,851, filed on Oct. 10, 2018.

(51) Int. Cl. A61H 5/00; A61H 9/00

(52) U.S. Cl. CPC A61H 5/00; A61H 9/0057; A61H 9/0007; A61H 2230/255; A61H 2201/5025; A61H 2201/5071; A61H 2205/024

(57) ABSTRACT

An apparatus to treat, inhibit, or prevent an indication of a headache symptom in a patient can include a cover, sized and shaped to fit over an eye of the patient to define a cavity between the cover and an anterior surface of the eye when the cover is located over the patient eye. The apparatus can include a pressure source, in communication with the cavity, capable of applying non-ambient cavity pressure to the cavity. The apparatus can include control circuitry, in communication with the pressure source, the control circuitry storing or otherwise configured to define a target cavity pressure value specified to treat, inhibit, or prevent the headache symptom, and configured to control the pressure source to adjust the cavity pressure toward the target cavity pressure value to treat, inhibit, or prevent the headache symptom.

### CLAIM OF PRIORITY

This patent application is a continuation from International Application No. PCT/US2019/055515, entitled “Apparatus and Methods to Treat Headaches”, filed Oct. 10, 2019, published as WO 2020/077032, which claims the benefit of priority of U.S. Provisional Patent Application Ser. No. 62/743,851 to John Berdahl entitled “Apparatus and Methods for Treating Headaches,” filed on Oct. 10, 2018, all of which are hereby incorporated by reference their entirety.

### BACKGROUND

Headache is a multi-factorial condition that afflicts millions of patients worldwide.

Liu U.S. Pat. No. 7,122,013 mentions a device for massaging eyes.

Lin U.S. Pat. No. 7,637,878 mentions an eye massaging device.

Lin U.S. Publication No. 20180042805 mentions an eye massager to massage acupuncture points.

### SUMMARY

Headache can be described as pain perceived in at least one of the head or neck region of an individual which can be caused by a variety of conditions and triggering stimuli. A primary goal of headache care can be adjustment or relief of an indication of a headache symptom as perceived by a patient. A headache can include at least one of an acute headache condition, such as a headache that can persist for a period of time measured in seconds, minutes, or days, or a chronic headache condition, such as a headache that can persist for a period of time measured in days, weeks, months, or years.

The present inventors have recognized, among other things, that there is a need in the art for apparatus and methods to adjust patient perception of an indication of a headache symptom, such as to treat, inhibit, or prevent the headache symptom. Adjustment of the indication of a headache symptom can include relief from the headache symptom, such as a reduction in pain perceived by the patient due to the headache condition.

An apparatus to treat, inhibit, or prevent an indication of a headache symptom in a patient can include a cover, sized and shaped to fit over an eye of the patient to define a cavity between the cover and an anterior surface of the eye when the cover is located over the patient eye. The apparatus can include a pressure source, in communication with the cavity, capable of applying non-ambient cavity pressure to the cavity. The apparatus can include control circuitry, in communication with the pressure source, the control circuitry storing, receiving, or otherwise configured to define a target cavity pressure value specified to treat, inhibit, or prevent the headache symptom, and configured to control the pressure source to adjust the cavity pressure toward the target cavity pressure value to adjust, such as treat, inhibit, or prevent, the headache symptom.

An overview of certain non-limiting aspects of the present subject matter is provided below.

Aspect 1 can include or use subject matter (such as an apparatus, a system, a device, a method, a means for performing acts, or a device readable medium including instructions that, when performed by the device, can cause the device to perform acts), such as an apparatus to treat, inhibit, or prevent an indication of a headache symptom in a patient including a cover, sized and shaped to fit over a patient eye to define a cavity between the cover and the patient, the cover capable of applying and retaining a non-ambient cavity pressure in contact with the patient to treat, inhibit, or prevent the headache symptom.

Aspect 2 can include or use or can optionally be combined with the subject matter of Aspect 1 to optionally include or use a positive pressure cavity check valve configured to release positive gauge pressure from the cavity and a negative pressure cavity check valve configured to regulate cavity pressure toward a target cavity pressure.

Aspect 3 can include or use or can optionally be combined with the subject matter of one or any combination of Aspects 1 or 2 to optionally include or use an apparatus including a pressure source, in communication with the cavity, capable of applying non-ambient cavity pressure to the patient to treat, inhibit, or prevent the headache symptom.

Aspect 4 can include or use or can optionally be combined with the subject matter of one or any combination of Aspects 1 through 3 to optionally include or use control circuitry, in communication with the pressure source, the control circuitry configured to receive a target cavity pressure level to control the pressure source to adjust the cavity pressure toward the target cavity pressure level to treat, inhibit, or prevent the headache symptom.

Aspect 5 can include or use or can optionally be combined with the subject matter of one or any combination of Aspects 1 through 4 to optionally include or use the target cavity pressure, wherein the target cavity pressure level includes a blood flow target cavity pressure level.

Aspect 6 can include or use or can optionally be combined with the subject matter of one or any combination of Aspects 1 through 5 to optionally include or use an ocular blood flow sensor, in communication with the control circuitry, to sense an indication of ocular blood flow in the patient eye and wherein the control circuitry includes blood flow pressure feedback control circuitry to adjust the cavity pressure toward the blood flow target cavity pressure level.

Aspect 7 can include or use or can optionally be combined with the subject matter of one or any combination of Aspects 1 through 6 wherein the target cavity pressure level includes an applied force target cavity pressure level.

Aspect 8 can include or use or can optionally be combined with the subject matter of one or any combination of Aspects 1 through 7 to optionally include or use a force sensor, in communication with the control circuitry, to sense an indication of force applied by the cover to patient tissue and wherein the control circuitry includes applied force pressure feedback control circuitry to adjust the cavity pressure toward the applied force target cavity pressure level.

Aspect 9 can include or use or can optionally be combined with the subject matter of one or any combination of Aspects 1 through 8 wherein the control circuitry includes sweep circuitry configured to sequentially vary non-ambient pressure level applied to the cavity in a pressure range to identify the target cavity pressure value, the pressure range defined by a first pressure level and a second pressure level, wherein the second pressure level is greater than the first pressure level.

Aspect 10 can include or use or can optionally be combined with the subject matter of one or any combination of Aspects 1 through 9 wherein the sweep circuitry is configured to sequentially vary non-ambient pressure from the first pressure level to the second pressure level.

Aspect 11 can include or use or can optionally be combined with the subject matter of one or any combination of Aspects 1 through 10 wherein the sweep circuitry is configured to sequentially vary non-ambient pressure from the second pressure level to the first pressure level.

Aspect 12 can include or use or can optionally be combined with the subject matter of one or any combination of Aspects 1 through 11 wherein the control circuitry includes patient input circuitry configured to receive an indication of an indication of a headache symptom from a patient.

Aspect 13 can include or use or can optionally be combined with the subject matter of one or any combination of Aspects 1 through 12 to optionally include or use a patient input interface, in communication with the patient input circuitry, configured to receive the indication of the headache symptom from the patient.

Aspect 14 can include or use or can optionally be combined with the subject matter of one or any combination of Aspects 1 through 13 wherein the patient input interface includes a fob device in communication with the control circuitry.

Aspect 15 can include or use or can optionally be combined with the subject matter of one or any combination of Aspects 1 through 14 wherein the patient input interface includes an app running on a mobile device, the app in communication with the control circuitry.

Aspect 16 can include or use or can optionally be combined with the subject matter of one or any combination of Aspects 1 through 15 wherein the control circuitry includes memory circuitry configured to store an indication of activity associated with the apparatus for use in at least one of programming the apparatus or monitoring patient use of the apparatus.

Aspect 17 can include or use or can optionally be combined with the subject matter of one or any combination of Aspects 1 through 16 to optionally include or use an anterior plate attached to the cover and configured to contact patient tissue proximal to at least a portion of an anterior portion of the skull including a patient trigeminal nerve.

Aspect 18 can include or use or can optionally be combined with the subject matter of one or any combination of Aspects 1 through 17 wherein the anterior plate is configured to apply pressure to the patient tissue, wherein the applied pressure is based upon non-ambient pressure applied to the cavity.

Aspect 19 can include or use or can optionally be combined with the subject matter of one or any combination of Aspects 1 through 18 wherein the control circuitry is configured to adjust the non-ambient pressure applied to the cavity toward a headache target cavity pressure.

Aspect 20 can include or use or can optionally be combined with the subject matter of one or any combination of Aspects 1 through 19 to optionally include or use a posterior plate attached to the cover and configured to contact patient tissue proximal to at least a portion of a posterior portion of the skull including a patient occipital nerve.

Aspect 21 can include or use or can optionally be combined with the subject matter of one or any combination of Aspects 1 through 20 wherein the posterior plate is configured to apply pressure to the patient tissue, wherein the applied pressure is based upon non-ambient pressure applied to the cavity.

Aspect 22 can include or use subject matter (such as an apparatus, a system, a device, a method, a means for performing acts, or a device readable medium including instructions that, when performed by the device, can cause the device to perform acts), or can optionally be combined with the subject matter of one or any combination of Aspects 1 through 21 to optionally include or use a method of using an apparatus to treat, inhibit, or prevent an indication of a headache symptom experienced by a patient, the method including forming a cavity over a patient eye; and pressurizing the cavity to generate a force against the patient to treat, inhibit or prevent the indication of the headache symptom.

Aspect 23 can include or use or can optionally be combined with the subject matter of one or any combination of Aspects 1 through 22 to optionally include or use the method wherein forming a cavity includes locating a cover over the patient eye and pressurizing the cavity includes, applying an external force to compress the cover against the patient, and releasing the external force to create a negative gauge pressure in the cavity.

Aspect 24 can include or use or can optionally be combined with the subject matter of one or any combination of Aspects 1 through 23 to optionally include or use the method including applying a non-ambient pressure to a cavity defined by a cover over the eye of the patient, wherein the patient has a history of experiencing the headache symptom; and adjusting the non-ambient pressure applied to the cavity toward a headache target cavity pressure level specified to treat, inhibit, or prevent the headache symptom.

Aspect 25 can include or use or can optionally be combined with the subject matter of one or any combination of Aspects 1 through 24 to optionally include or use the method wherein the patient with a history of experiencing the headache symptom is experiencing the headache symptom.

Aspect 26 can include or use or can optionally be combined with the subject matter of one or any combination of Aspects 1 through 17 to optionally include or use the method wherein the cover includes an anterior plate and the method comprises locating the anterior plate in contact with patient tissue proximal to at least a portion of a patient trigeminal nerve.

Aspect 27 can include or use or can optionally be combined with the subject matter of one or any combination of Aspects 1 through 26 to optionally include or use the method wherein adjusting non-ambient pressure includes adjusting non-ambient pressure toward a headache target cavity pressure.

Aspect 28 can include or use or can optionally be combined with the subject matter of one or any combination of Aspects 1 through 27 to optionally include or use the method wherein the cover includes a posterior plate and the method comprises locating the posterior plate in contact with patient tissue proximal to the occipital nerve.

Aspect 29 can include or use or can optionally be combined with the subject matter of one or any combination of Aspects 1 through 28 to optionally include or use the method wherein adjusting non-ambient pressure includes adjusting non-ambient pressure toward a headache target cavity pressure.

Aspect 30 can include or use subject matter (such as an apparatus, a system, a device, a method, a means for performing acts, or a device readable medium including instructions that, when performed by the device, can cause the device to perform acts), such as an apparatus to affect a headache symptom in a patient, wherein the apparatus can include a cover sized and shaped to fit over an eye of the patient to define a cavity between the cover and an anterior surface of the eye, the cover including a patient interface surface with a protuberance located between the cover and the patient, the protuberance configured to apply a force to the patient to affect the headache symptom. A pressure source, in communication with the cavity, configured to apply non-ambient pressure to the cavity, wherein the protuberance force is variable based on the non-ambient pressure applied to the cavity.

Aspect 31 can include or use or can optionally be combined with the subject matter of Aspect 30 wherein the protuberance includes at least one of a positive protuberance or a negative protuberance.

Aspect 32 can include or use or can optionally be combined with the subject matter of one or any combination of Aspects 30 or 31 wherein the protuberance includes the positive protuberance.

Aspect 33 can include or use or can optionally be combined with the subject matter of one or any combination of Aspects 30 through 32 wherein the protuberance includes the negative protuberance.

Aspect 34 can include or use or can optionally be combined with the subject matter of one or any combination of Aspects 30 through 33 wherein at least one of the positive protuberance or the negative protuberance is configured to be movable on the patient interface surface.

Aspect 35 can include or use or can optionally be combined with the subject matter of one or any combination of Aspects 30 through 34 wherein the patient interface surface is configured to vary the temperature of the patient interface surface.

Aspect 36 can include or use or can optionally be combined with the subject matter of one or any combination of Aspects 30 through 35 wherein the temperature can be varied by at least one of increasing the temperature of the patient interface surface or decreasing the temperature of the patient interface surface.

Aspect 37 can include or use or can optionally be combined with the subject matter of one or any combination of Aspects 30 through 36 wherein the patient interface surface is configured to transmit energy to the patient in a frequency range of about 10 Hz to about 100 kHz.

Aspect 38 can include or use or can optionally be combined with the subject matter of one or any combination of Aspects 30 through 37 including an anterior plate attached to the cover and configured to contact patient tissue surrounding at least a portion of the trigeminal nerve.

Aspect 39 can include or use or can optionally be combined with the subject matter of one or any combination of Aspects 30 through 38 wherein the anterior plate is configured to apply contact pressure to tissue surrounding at least a portion of the trigeminal nerve.

Aspect 40 can include or use or can optionally be combined with the subject matter of one or any combination of Aspects 30 through 39 wherein the anterior plate is configured to vary the temperature of the anterior plate interface surface.

Aspect 41 can include or use or can optionally be combined with the subject matter of one or any combination of Aspects 30 through 40 wherein the anterior plate is configured to transmit energy to the patient in a frequency range of about 10 Hz to about 100 kHz.

Aspect 42 can include or use or can optionally be combined with the subject matter of one or any combination of Aspects 30 through 41 including a posterior plate attached to the cover and configured to contact tissue surrounding at least a portion of the occipital nerve.

Aspect 43 can include or use or can optionally be combined with the subject matter of one or any combination of Aspects 30 through 42, wherein the posterior plate is configured to apply contact pressure to the patient tissue surrounding at least a portion of the occipital nerve.

Aspect 44 can include or use or can optionally be combined with the subject matter of one or any combination of Aspects 30 through 43 wherein the posterior plate is configured to transmit energy to the patient in a frequency range of about 10 Hz to about 100 kHz.

Aspect 45 can include or use or can optionally be combined with the subject matter of one or any combination of Aspects 30 through 44 to optionally include or use the method including locating a cover over a patient eye to form a cavity between the cover and an anterior surface of the patient eye, the cover including a patient interface surface with a protuberance between the cover and the patient, the protuberance configured to apply a protuberance force to the patient; and applying non-ambient pressure to the cavity to vary the protuberance force to affect the headache symptom.

Aspect 46 can include or use or can optionally be combined with the subject matter of one or any combination of Aspects 30 through 45 to optionally include or use the method wherein locating the cover includes adjusting the location of at least one of a positive protuberance or a negative protuberance on the patient interface surface.

Aspect 47 can include or use or can optionally be combined with the subject matter of one or any combination of Aspects 30 through 46 to optionally include or use the method wherein the apparatus includes an anterior plate attached to the cover and locating the cover includes locating the anterior plate in contact with patient tissue surrounding at least a portion of the trigeminal nerve.

Aspect 48 can include or use or can optionally be combined with the subject matter of one or any combination of Aspects 30 through 47 to optionally include or use the method wherein applying non-ambient pressure includes applying contact pressure to patient tissue surrounding at least a portion of the trigeminal nerve with the anterior plate.

Aspect 49 can include or use or can optionally be combined with the subject matter of one or any combination of Aspects 30 through 48 to optionally include or use the method wherein the apparatus includes a posterior plate attached to the cover and locating the cover includes locating the posterior plate in contact with patient tissue surrounding at least a portion of the occipital nerve.

Aspect 50 can include or use or can optionally be combined with the subject matter of one or any combination of Aspects 30 through 49 to optionally include or use the method wherein applying non-ambient pressure includes applying contact pressure to patient tissue surrounding at least a portion of the occipital nerve with the posterior plate.

Aspect 51 can include or use subject matter (such as an apparatus, a system, a device, a method, a means for performing acts, or a device readable medium including instructions that, when performed by the device, can cause the device to perform acts), such as an apparatus to affect a headache symptom in a patient, the apparatus including a cover sized and shaped to fit over an eye of the patient to define a cavity between the cover and an anterior surface of the eye; and control circuitry, configured to regulate a pressure source in communication with the cavity and an adjunct device in communication with the patient to affect the headache symptom.

Aspect 52 can include or use or can optionally be combined with the subject matter of Aspect 51 wherein the adjunct device includes at least one of a trigeminal energy transfer device, an occipital energy transfer device, or a peripheral nerve energy transfer device.

Aspect 53 can include or use or can optionally be combined with the subject matter of one or any combination of Aspects 51 or 52 wherein the adjunct device is the trigeminal energy transfer device with a patient interface surface located against tissue proximal to the patient trigeminal nerve.

Aspect 54 can include or use or can optionally be combined with the subject matter of one or any combination of Aspects 51 through 53 wherein the patient interface surface is configured to adjust temperature of the patient interface surface including at least one of increasing the temperature of the patient surface interface or decreasing the temperature of the patient surface interface.

Aspect 55 can include or use or can optionally be combined with the subject matter of one or any combination of Aspects 51 through 54 wherein the patient interface surface is configured to transmit energy to the patient in a frequency range of about 10 Hz to about 100 kHz.

Aspect 56 can include or use or can optionally be combined with the subject matter of one or any combination of Aspects 51 through 55 wherein the frequency range is about 10 Hz to about 500 Hz.

Aspect 57 can include or use or can optionally be combined with the subject matter of one or any combination of Aspects 51 through 56 wherein the frequency range is about 500 Hz to about 10 kHz.

Aspect 58 can include or use or can optionally be combined with the subject matter of one or any combination of Aspects 51 through 57 wherein the frequency range is about 10 kHz to about 50 kHz.

Aspect 59 can include or use or can optionally be combined with the subject matter of one or any combination of Aspects 51 through 58 wherein the adjunct device is the occipital energy transfer device with a patient interface surface located against tissue proximal to the patient occipital nerve.

Aspect 60 can include or use or can optionally be combined with the subject matter of one or any combination of Aspects 51 through 59 wherein the patient interface surface is configured to adjust temperature of the patient interface surface including at least one of increasing the temperature of the patient surface interface or decreasing the temperature of the patient interface surface.

Aspect 61 can include or use or can optionally be combined with the subject matter of one or any combination of Aspects 51 through 60 wherein the patient interface surface is configured to transmit energy to the patient in a frequency range of about 10 Hz to about 100 kHz.

Aspect 62 can include or use or can optionally be combined with the subject matter of one or any combination of Aspects 51 through 61 wherein the frequency range is about 10 Hz to about 500 Hz.

Aspect 63 can include or use or can optionally be combined with the subject matter of one or any combination of Aspects 51 through 62 wherein the frequency range is about 500 Hz to about 10 kHz.

Aspect 64 can include or use or can optionally be combined with the subject matter of one or any combination of Aspects 51 through 63 wherein the frequency range is about 10 kHz to about 50 kHz.

Aspect 65 can include or use or can optionally be combined with the subject matter of one or any combination of Aspects 51 through 64 wherein the adjunct device is the peripheral nerve energy transfer device with a patient interface surface located against tissue proximal to a peripheral nerve of the patient.

Aspect 66 can include or use subject matter (such as an apparatus, a system, a device, a method, a means for performing acts, or a device readable medium including instructions that, when performed by the device, can cause the device to perform acts), or can optionally be combined with the subject matter of one or any combination of Aspects 51 through 65 to optionally include or use a method of using an apparatus to affect a headache symptom, the apparatus including a cover sized and shaped to fit over an eye of a patient to define a cavity between the cover and an anterior surface of the eye and control circuitry to regulate a pressure source in communication with the cavity and an adjunct device in communication with the patient, the method including adjusting non-ambient pressure in the cavity toward a headache target cavity pressure level with the pressure source to affect the headache symptom, and adjusting energy applied to the patient toward a headache target energy level with the adjunct device to further affect the headache symptom.

Aspect 67 can include or use or can optionally be combined with the subject matter of one or any combination of Aspects 51 through 66 to optionally include or use the method wherein the adjunct device includes an anterior plate configured to contact patient tissue proximal to at least a portion of a patient trigeminal nerve and adjusting energy includes applying pressure to the patient tissue toward a trigeminal nerve headache target contact pressure with the anterior plate.

Aspect 68 can include or use or can optionally be combined with the subject matter of one or any combination of Aspects 51 through 67 to optionally include or use the method wherein applying pressure to the patient to the patient tissue includes applying pressure based upon the non-ambient pressure level in the cavity.

Aspect 69 can include or use or can optionally be combined with the subject matter of one or any combination of Aspects 51 through 68 to optionally include or use the method wherein the adjunct device includes a posterior plate configured to contact patient tissue proximal to at least a portion of a patient occipital nerve and adjusting energy includes applying pressure to the patient tissue toward an occipital nerve headache-relief target contact pressure with the posterior plate.

Aspect 70 can include or use or can optionally be combined with the subject matter of one or any combination of Aspects 51 through 69 to optionally include or use the method wherein applying pressure to the patient tissue includes applying pressure based upon the non-ambient pressure level in the cavity.

Aspect 71 can include or use or can optionally be combined with the subject matter of one or any combination of Aspects 51 through 70 to optionally include or use the method wherein the adjunct device includes a peripheral energy transfer device configured to contact patient tissue proximal to at least a portion of a patient peripheral nerve and adjusting energy includes applying stimulation energy to the patient tissue toward a peripheral nerve headache target energy level with the peripheral energy transfer device.

Aspect 72 can include or use or can optionally be combined with the subject matter of one or any combination of Aspects 51 through 71 to optionally include or use the method wherein the adjunct device includes an energy transfer device configured to contact patient tissue proximal to at least a portion of a patient peripheral nerve and adjusting energy includes applying stimulation energy to the patient tissue toward a peripheral nerve headache target energy level with the peripheral energy transfer device.

Each of these non-limiting examples can stand on its own or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an example of an apparatus to control an environment over a patient eye.

FIGS. 2A and 2B show a sectional side view of an example of a positive pressure cavity check valve 181 located in a cover, such as a flapper valve configured to control pressure in the cavity to a positive target cavity pressure level.

FIGS. 3A and 3B show a sectional side view of an example of a negative pressure cavity check valve located in a cover, such as a flapper valve configured to control pressure in the cavity to a negative target cavity pressure level.

FIG. 4 shows a sectional side view of an example of a check valve assembly, such as a flapper check valve assembly in an open position.

FIGS. 5A and 5B shows an example of a sectional side view of a positive pressure cavity check valve 181 and a negative pressure cavity check valve located in a cover.

FIG. 6 shows an illustration of acupressure points on facial tissue.

FIGS. 7A and 7B show an example of a protuberance and a recess located on the patient interface surface.

FIG. 8 shows an example of the cover including an anterior plate.

FIG. 9 shows an example of the cover including a posterior plate.

FIG. 10 shows an illustration of posterior acupressure points on a patient.

FIG. 11 shows an example of an apparatus that can control an eye environment over a patient eye, such as at least one of a left eye environment over the left patient eye or a right eye environment over the right patient eye.

FIG. 12 shows an example of an apparatus that can independently control a left eye environment over a left eye of a patient and a right eye environment over a right eye of the patient, such as with a single pressure source.

FIG. 13 shows an example method for using the apparatus to adjust patient perception of an indication of a headache symptom.

FIG. 14 shows an example block diagram of an example computing machine that can be used as control circuitry.

### DETAILED DESCRIPTION

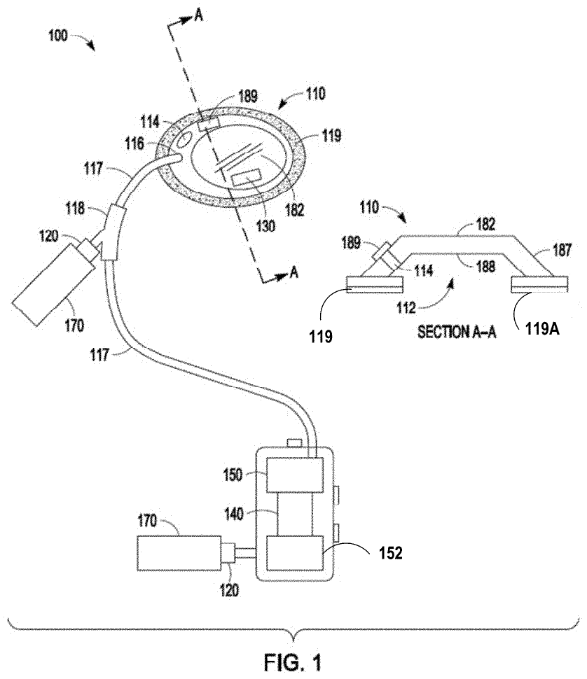

FIG. 1 shows an example of an apparatus 100, such as to stimulate patient tissue to adjust a headache symptom experienced by a patient, such as through control of an eye environment over a patient eye. Stimulation of patient tissue can include at least one of the absorption of one or more therapeutic components into patient tissue, such as through adjustment of fluid composition in the eye environment, or the application of force to patient tissue, such as through adjustment of non-ambient pressure associated with the eye environment. In an example, the patient tissue can include any tissues of the patient affected by the eye environment, such as an anterior surface of the patient eye or patient skin exposed to the eye environment. The apparatus 100 can include a cover 110, a fluid regulator 120, a sensor.

The cover 110 can be sized and shaped to surround the patient eye and be spaced from the eye, such as without contacting the eye including the anterior surface of the eye. The cover 110 can be sized and shaped to surround and cover both patient eyes, such as the left eye and the right eye of a patient. In an example, the cover 110 can include a mask, such as a cover 110 similar in shape and function to a diving or snorkeling mask that can cover both the left eye and the right eye of the patient.

The cover 110 can include a lens portion 182 to allow a patient to see outward through the cover 110 or to allow observation of the eye, such as exterior structures of the eye including the cornea or intraocular structures of the eye including the retina, inward through the cover 110. The lens portion 182 can serve as a corrective lens for the patient, such as to correct an astigmatism of the eye. The lens portion 182 can include a lens blank, such as an A8 lens blank, that can be shaped as a prescription lens for the patient, such as to correct for refractive error in the eye.

The lens portion 182 can include a replaceable lens portion 182, such as a first lens portion in the apparatus 100 can be interchanged with a second lens portion, such as to change the lens magnification presented to the patient. In an example, lens magnification can be selected to allow for examination of the intraocular space of the eye including assessment of the retina and the choroid, such as for at least one of diagnostic or treatment purposes. The lens magnification can be selected to enhance the examination of the eye, such as to focus the lens portion 182 to enhance visualization of a portion of the eye. The inner surface of the lens portion 182 can be treated, such as with an anti-fog coating to prevent condensation from obscuring the view of the patient.

The lens portion 182 can be configured to control ambient light entering the cavity 112, such as to adjust or relieve a headache symptom. The lens portion 182 can include tinting, such as an auto-tinting lens to reduce the intensity of light to adjust or relieve the headache symptom.

The cover 110 can define an enclosed cavity 112, such as when the cover 110 is placed over the eye and against the patient. In an example, a peripheral edge of the cover 110 placed over the eye can contact at least a portion of patient tissue proximal to the eye socket, such as to form the cavity 112. The cavity 112 can define an enclosed cavity 112 over both eyes, such as when the cover 110 includes a mask located over the left and right patient eyes.

The cavity 112 can include a spatial volume, such as the spatial volume defined between an inner surface 188 of the cover 110, and patient tissue, such as including an anterior surface of the patient eye. The cavity 112 can contain a working fluid, such as a liquid or gaseous fluid, that can form at least part of an eye environment in contact with the patient tissue, such as including the anterior surface of the patient eye.

The cover 110 can include a first port 114. The first post 114 can be located in a surface of the cover 110, such as the first port 114 can extend from an outer surface 187 of the cover 110 to an inner surface 188 of the cover 110, to allow access to the eye environment in the cavity 112. The first port 114 can include a septum, such as a flexible septum located over the first port 114 to isolate the cavity 112 from the surrounding environment. The flexible septum can maintain a gauge pressure in the cavity 112, such as at least one of a positive or negative gauge pressure.

The flexible septum can include a resealable septum, such as a septum formed from a self-healing material including a self-sealing polymer material that can allow the insertion and withdrawal of instruments through the septum into the cavity 112 while maintaining a gauge pressure in the cavity 112. In an example, the resealable septum can allow a hypodermic needle to be inserted and withdrawn through the resealable septum while maintaining a gauge pressure (e.g. a positive or negative gauge pressure) in the cavity 112. For example, the resealable septum can allow for a hypodermic needle to be placed in proximity of the eye, such as to place a therapeutic fluid in contact with the eye, while maintaining a gauge pressure in the cavity 112.

The flexible septum can include a measurement septum, such as a septum to allow a sensor, such as the sensor 130, to sense an indication of the eye environment in the cavity 112 without contacting the eye environment. In an example, a pressure sensor can be in contact with the measurement septum covering the first port 114 of the cover 110, such as to sense an indication of working fluid pressure in the cavity 112 through the measurement septum.

The cover 110 can include a second port 116, such as extending from an outer surface 187 of the cover 110 to an inner surface 188 of the cover 110. In an example, the second port 116 can place the cavity 112 in communication with the pressure source 150, such as with a conduit 117.

The apparatus 100 can include a cavity check valve 189. The cavity check valve 189 can be located on the apparatus 100 in communication with the cavity 112, such as on at least one of the cover 110 including any surface of the cover 110, the conduit 117, the control circuitry 140, or the pressure source 150. In an example, the cavity check valve 189 can be located in proximity to, such as in, on, or over, the first port 114.

The cavity check valve 189 can limit fluid pressure, such as in the cavity 112. In an example, the cavity check valve 189 can be used as a safety valve, such as to ensure that pressure in the cavity 112 will not exceed a cavity pressure level that could damage the patient eye. In an example, the cavity check valve 189 can limit pressure in the cavity 112, such as to a target cavity pressure level.

The cavity check valve 189 can include a cracking pressure, such as a characteristic of the cavity check valve 189 that can control initiation of fluid flow through the valve. In an example, the cracking pressure can describe an inlet pressure level of the cavity check valve 189, such as an inlet pressure level at which a fluid can initiate flow through the cavity check valve 189. Fluid pressure in the cavity 112 can be limited to the target cavity pressure level, such as by selecting or setting the cracking pressure of the cavity check valve 189 to equal the target cavity pressure level. In an example, when the fluid pressure in the cavity 112 is less than the cracking pressure of the cavity check valve 189, the cavity check valve 189 can assume a closed state, such as to prevent the flow of fluid from the cavity 112 to the surrounding atmosphere. When the fluid pressure in the cavity 112 is equal to or greater than the cracking pressure of the cavity check valve 189, the cavity check valve 189 can assume an open state, such as to allow a flow of fluid from the cavity 112 to the surrounding atmosphere.

The cavity check valve 189 can include a passive cavity check valve, such as a flapper valve or a poppet valve. The cracking pressure of the passive cavity check valve can be adjusted, such as by changing the dimensions of the passive cavity check valve or components of the passive cavity check valve. In an example, the cracking pressure of a flapper cavity check valve can be adjusted, such as by changing at least one of the flapper check valve dimensions (e.g., length, width, thickness), the flapper check valve constituent material (e.g. type of material, durometer of material, single or multi-ply material, stiffness of valve), or the flapper check valve hinge. In an example, the cracking pressure of a poppet cavity check valve can be adjusted, such as by changing at least one of the poppet valve dimensions (e.g., spring stiffness, poppet diameter).

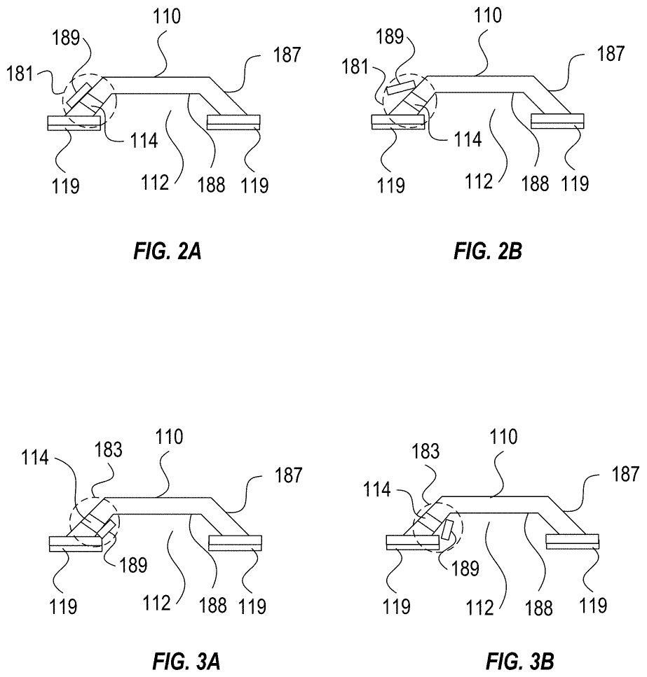

FIGS. 2A and 2B show a sectional side view of an example of a positive pressure cavity check valve 181 located in a cover 110, such as a flapper valve configured to control pressure in the cavity 112 to a positive target cavity pressure level. FIG. 2A shows the positive pressure cavity check valve 181 in the closed position. FIG. 2B shows the positive pressure cavity check valve 181 in the open position. The positive target cavity pressure level can be specified, such as by a medical professional to treat, inhibit, or prevent an eye condition. The positive pressure cavity check valve 181 can be located on the cover 110, such as the outer surface 187 of the cover 110 to allow positive pressure working fluid in the cavity 112 at a pressure greater than the cracking pressure of the check valve to flow from the cavity 112 to the surrounding environment.

As shown in FIG. 2A, the cavity check valve 189 can assume a closed position, such as fluid cannot pass from the cavity 112 through the cavity check valve 189 to the surrounding environment. In the closed position, the apparatus 100 can support a positive gauge pressure in the cavity 112, such as a positive gauge pressure level less than the positive target cavity pressure level. The positive target cavity pressure level can include a positive safety pressure threshold, such as threshold pressure above which damage to the patient eye can occur. The positive target cavity pressure level can be controlled, such as by setting the cracking pressure of the positive pressure cavity check valve 181 to equal the positive target cavity pressure level.

As shown in FIG. 2B, the cavity check valve 189 can assume an open position, such as fluid can pass from the cavity 112 through the cavity check valve 189 to the surrounding environment, such as when the positive gauge pressure in the cavity 112 is equal to or greater than the positive target cavity pressure level. In the open position, the apparatus 100 can limit the positive gauge pressure environment in the cavity 112 to a pressure level approximately equal to the positive target cavity pressure level, such as to protect the eye from excessive fluid pressure.

FIGS. 3A and 3B show a sectional side view of an example of a negative pressure cavity check valve located in a cover 110, such as a flapper valve configured to control pressure in the cavity 112 to a negative target cavity pressure level. FIG. 3A shows the negative pressure cavity check valve in the closed position. FIG. 3B shows the negative pressure cavity check valve in the open position. The negative target cavity pressure level can be specified, such as by a medical professional to treat, inhibit, or prevent an eye condition. The negative pressure cavity check valve can be located on the cover 110, such as the inner surface 188 of the cover 110 to allow fluid from the surrounding environment to flow into the cavity 112 from the surrounding environment.

As shown in FIG. 3A, the cavity check valve 189 can assume a closed position, such as ambient fluid cannot pass into the cavity 112 through the cavity check valve 189 from the surrounding environment. In the closed position, the apparatus 110 can support a negative gauge pressure environment in the cavity 112, such as a negative gauge pressure level greater than the negative target cavity pressure level. The negative target cavity pressure level can include a negative safety pressure threshold, such as threshold pressure below which damage to the patient eye can occur. The negative target cavity pressure level can be controlled, such as by setting the cracking pressure of the negative pressure cavity check valve to equal the negative target cavity pressure level.

As shown in FIG. 3B, the cavity check valve 189 can assume an open position, such as ambient fluid can pass into the cavity 112 through the cavity check valve 189 from the surrounding environment, such as when the negative gauge pressure in the cavity 112 is equal to or less than the negative target cavity pressure level. In the open position, the apparatus 100 can limit the negative gauge pressure environment in the cavity 112 to a pressure level approximately equal to the negative target cavity pressure level, such as to prevent possible damage to the eye by excessive working fluid pressure.

As the patient eye condition changes, such as improves or degrades, a medical professional can adjust the prescribed treatment regimen, such as to change at least one of the positive target cavity pressure level or the negative target cavity pressure level. To adjust a target pressure level, the assembly 100 can include an adjustable valve. In an example, an adjustable valve can include a replaceable valve, such as a replaceable check valve assembly.

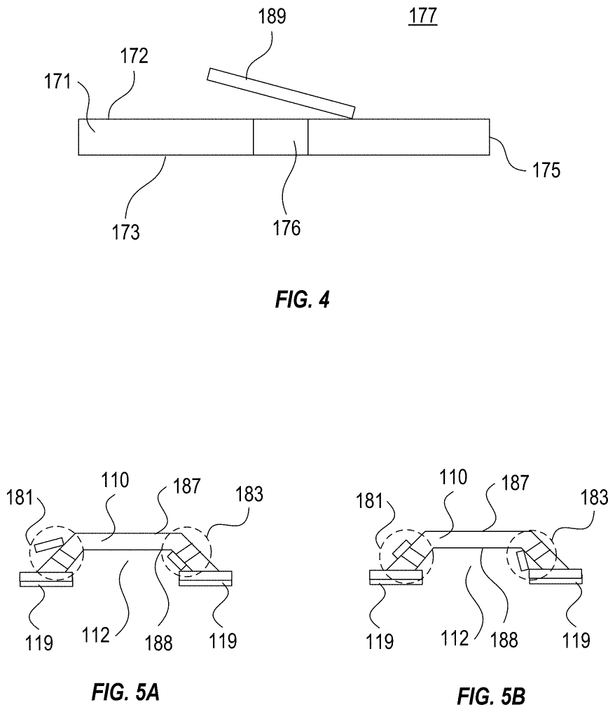

FIG. 4 shows a sectional side view of an example of a check valve assembly 177, such as a flapper check valve assembly in an open position. The apparatus 100 can include a check valve assembly 177, such as a replaceable check valve assembly 177 to adjust the target cavity pressure level in the cavity 112. In an example, the apparatus 100 with a first check valve assembly including a first cavity check valve with a first cracking pressure set to a first target pressure level, can be replaced with a second check valve assembly including a second cavity check valve with a second cracking pressure set to a second target pressure level. Changing from the first check valve assembly to the second check valve assembly can realize a change in pressure applied to the patient eye, such as a change in pressure specified in a prescribed patient treatment regimen, such as including a change in target cavity pressure level.

The cavity check valve assembly 177 can include a base 171 with a first side 172, a second side 173 parallel to the first side 172, a base periphery 175 extending from the first side 172 to the second side 173, a base port 176 extending through the base 171 from the first side 172 to the second side 173, and a cavity check valve 189 located on the first side 172 over the base port 176, such as at least a portion of the base port 176. The cavity check valve assembly 177 can be located in the apparatus 100, such as in the cover 110 so that the base periphery 175 can be in contact with the cover 110, such as at least a portion of the surface of the port 114.

The check valve assembly 177 can be located on the apparatus 100 in communication with the cavity 112, such as on at least one of the cover 110 including any surface of the cover 110, the conduit 117, the control circuitry 140, or the pressure source 150. The cavity check valve assembly 177 can be located in contact with the cover 110, such as the base periphery 175 can be in contact with at least a portion of the cover 110, such as at least one of the surface of the port 114, the outer surface 187, or the inner surface 188.

The cavity check valve assembly 177, such as a positive pressure check valve assembly, can be configured to control pressure in the cavity 112 to a positive target cavity pressure level, such as the check valve assembly 177 can be located in the port 114 so that the cavity check valve 189 can be located outside of the cavity 112. The cavity check valve assembly 177, such as a negative pressure check valve assembly, can be configured to control pressure in the cavity 112 to a negative target cavity pressure level, such as the check valve assembly 177 can be located in the port 114 so that the cavity check valve 189 can be located inside the cavity 112.

FIGS. 5A and B show an example of a sectional side view of a positive pressure cavity check valve 181 and a negative pressure cavity check valve 183 located in a cover 110. The cover 110 can be located over the patient eye with the seal 119 in contact with the patient, such as to form the cavity 112 over the patient eye. The cavity 112 can be pressurized, such as to create a non-ambient pressure, such as at least one of a positive gauge pressure or a negative gauge pressure in contact with the patient, such as patient tissue.

In an example, the cavity 112 can be pressurized, such as by an external force applied to the cover 110. The external force can include a force separate from the apparatus 100, such as an external force applied by a hand of a user or a medical professional.

The external force can press the cover 110 against the patient tissue, such as to compress at least one of the seal 119 or patient tissue. Compression of at least one of the seal 119 or patient tissue can cause a reduction in cavity volume, such as to compress fluid in the cavity 112 resulting in increased cavity pressure, such as to create a positive gauge pressure in the cavity. The increased cavity pressure can be relieved, such as by releasing a quantity of fluid from the cavity 112 through at least one of the positive pressure cavity check valve 181 or leakage around or through the seal 119, such as to leave a reduced quantity of fluid remaining in the cavity 112.

The positive gauge pressure generated by the external force can be regulated, such as by selecting the cracking pressure of the positive pressure check valve 181 to equal a positive target cavity pressure level. A positive target cavity pressure level can be selected, such as to ensure that a negative gauge pressure exists in the cavity 112 after the external force is released. In an example, a positive target cavity pressure level can include a pressure level, such as equal to or less than the ambient pressure surrounding the cover 110. FIG. 5A shows the positive pressure check cavity check valve 181 in an open state, such as to regulate the pressure in the cavity 112 to the positive target cavity pressure level.

As the external force is released, at least one of the seal 119 or patient tissue can rebound from the compressed position, such as to cause an increase in cavity volume to decrease the pressure of the fluid remaining in the cavity 112, such as to create a negative gauge pressure in the cavity 112. The resulting negative gauge pressure can create a “suction” force in the cavity 112, such as to draw the cover 110 toward the patient, such as to generate an applied force against patient tissue. The applied force generated can act to stimulate the patient tissue, such as including patient tissue proximal to the applied force, such as to treat, inhibit, or prevent a headache symptom.

The magnitude of the force applied to the patient tissue can be regulated, such as by controlling the negative gauge pressure level in the cavity 112. In an example, the negative gauge pressure level can be regulated, such as by selecting the cracking pressure of the negative pressure cavity check valve 183 to equal a negative target cavity pressure level. The negative target cavity pressure level can be selected, such as to retain negative gauge pressure in the cavity 112 in a range of negative gauge pressure sufficient to treat, inhibit, or prevent the headache symptom. In an example, the range of negative gauge pressure can include a range, such as a range including at least one of a range of about −5 mmHg to about −15 mmHg, a range of about −15 mmHg to about −25 mmHg, a range of about −25 mmHg to about −35 mmHg, or a range of about −35 mmHg to about −45 mmHg.

The cover 110 can retain the working fluid against the patient, such as in contact with patient tissue including the anterior portion of the patient eye, to form the eye environment in the cavity 112. Exposure of the patient to the eye environment can stimulate patient tissue to adjust patient perception of an indication of a headache symptom, such as to treat, inhibit, or prevent the headache symptom. An indication of a headache symptom can include at least one of an indication of the presence of a headache symptom as perceived by the patient, such as to document the presence of the headache symptom for assessment purposes, an indication of the pain intensity of the headache symptom, such as the pain intensity perceived by the patient experiencing the headache symptom, an indication of the presence of a biomarker, such as the presence of a biomarker associated with the headache symptom and sensed by a sensor 130, or an indication of blood vessel caliber, such as ocular blood vessel caliber. In an example, adjusting patient perception of an indication of a headache symptom can include relieving pain experienced by the patient associated with the indication of the headache symptom.

Adjustment of the headache symptom can include exposure of the patient eye to the composition of the working fluid in the eye environment to stimulate the patient eye, such as to facilitate absorption of the working fluid including a therapeutic component of the working fluid into the eye. In an example, a therapeutic component can include at least one of a vasodilator or a vasoconstrictor, such as to treat, inhibit, prevent, or adjust patient perception of an indication of a headache symptom experienced by the patient.

A constituent fluid can include a substance capable of vasoconstriction, such as ocular blood vessel vasoconstriction. In an example, a vasoconstrictor can include at least one of an alpha-adrenoceptor agonist, a vasopressin analog, epinephrine, norepinephrine, phenylephrine, dopamine, dobutamine, or other migraine and headache medications, such as at least one of a serotonin 5-hydroxytryptamine agonist or a triptan.

A constituent fluid can include a substance capable of vasodilation, such as ocular blood vessel vasodilation. In an example, a vasodialator can include at least one of a combination of nitrogen and nitric oxide, such as the nitric oxide constituent can be absorbed through a surface of the eye to promote vasodilation of blood vessels to adjust, such as treat, inhibit, or prevent, an indication of a headache symptom.

The working fluid can be composed of one or more constituent fluids, such as a combination of one or more liquids or gases. A working fluid can include a combination of two constituent fluids, such as a combination of gaseous nitric oxide and gaseous carbon dioxide. A constituent fluid can include a therapeutic fluid, such as a component of the constituent fluid can be absorbed through the eye to inhibit, treat, or prevent a headache symptom.

A therapeutic fluid can include a gaseous therapeutic fluid, such as at least one of carbon dioxide (CO₂), oxygen (O₂), nitric oxide (NO), ozone (O₃), nitrogen (N₂), helium (He), hydrocarbons including fluorocarbons and perfluorocarbons, sulfur hexafluoride, cannabinoids including tetrahydrocannabinol (THC) and cannabidiol (CBD), a combination of two or more gaseous therapeutic fluids, or the like. In an example, a therapeutic gas can include a mixture of at least one of carbon dioxide, oxygen, or nitric oxide, such as to treat, inhibit, or prevent an indication of a headache symptom. In an example, a therapeutic gas can include a mixture of nitric oxide and oxygen including a mixture of 50% nitric oxide and 50% oxygen, a mixture of helium and oxygen (also known as heliox), and Medical Air including Medical Grade Air USP, such as to treat, inhibit, or prevent an indication of a headache symptom. In an example, a mixture of therapeutic gases can include a mixture of nitric oxide and oxygen, such as a mixture of 50% nitric oxide and 50% oxygen including gases from The BOC Group plc under the tradename ENTONOX, such as to treat an indication of a headache symptom. In an example, a combination of therapeutic gases can include a mixture of helium and oxygen, such as a mixture of 21% oxygen and 79% helium, also known as heliox, such as to treat an indication of a headache symptom. In an example, a combination of therapeutic gases can include a mixture of at least one of fluorine or chlorine, such as to treat an indication of a headache symptom. In an example, a combination of therapeutic gases can include at least one of a mixture with a volume fraction of oxygen less than ambient air, such as the mixture with less than about twenty-one percent volume fraction O₂, or a mixture with a volume fraction of oxygen greater than ambient air, such as the mixture with more than about twenty-one percent volume fraction O₂, such as to treat an indication of a headache symptom.

The eye environment can be used to characterize a physiological state of the patient eye, such as the eye environment can include physiological constituents including biomarkers emitted from the eye or patient tissue within the cavity 112. In an example, the presence of a headache or stimulation of the eye or patient tissue can cause the emissions of biomarkers, such as from the patient eye or patient tissue. Information sensed by the apparatus 100, such as biomarkers sensed from the working fluid in the cavity 112, can provide a medical professional with patient information, such as to diagnosis an eye condition associated with the patient eye or the presence, type, or severity of a headache.

A mechanism of action to trigger at least one of a headache or a headache symptom, can include decreased perfusion of the patient eye, such as decreased macular perfusion. In an example, the apparatus 100 can be used to sense an indication of blood flow in the patient eye, such as with a sensor 130, such as including a blood flow sensor. The control circuitry 140 can receive and process the sensed blood flow data, such as to determine a state of blood flow in the patient eye. A target cavity pressure, such as a blood flow target cavity pressure, can be calculated, such as based on the state of blood flow in the patient eye, and used to adjust pressure level in the cavity 112, such as to adjust the pressure level toward the blood flow target cavity pressure, such as to treat, inhibit, or prevent a headache symptom in the patient.

The eye environment can be defined by an environmental parameter, such as a characteristic of the working fluid in the cavity 112. An environmental parameter can include at least one of working fluid flow in the cavity 112, such as working fluid volumetric flow rate into or out of the cavity 112, working fluid humidity in the cavity 112, such as the relative humidity of the working fluid in the cavity 112, working fluid temperature in the cavity 112, working fluid pressure in the cavity 112 (e.g., cavity pressure), such as the working fluid gauge pressure in the cavity 112 and the ambient pressure of the environment surrounding the cavity, or working fluid composition in the cavity 112, such as working fluid composition measured by at least one of constituent fluid concentration or partial fluid pressure. An environmental parameter can include a parameter associated with the cover 110, such as at least one of tension in the anterior plate harness 194, tension in the posterior plate tether, or force applied to patient tissue, such as at the patient interface surface 119A.

Adjustment of the headache symptom can include exposure of the patient to non-ambient pressure associated with the eye environment in the cavity 112, such as to generate force against the patient tissue. As the cover 110 can be configured to contact patient tissue, the patient tissue can react the force applied by the cover 110 due to the eye environment and stimulate the patient tissue proximal to and in contact with the cover 110, such as to adjust patient perception of an indication of the headache symptom. In an example, the cover 110 can apply force to patient tissue due to non-ambient pressure in the cavity 112 and the force applied to patient tissue can be adjusted, such as by adjusting non-ambient pressure in the cavity 112. In an example, the working fluid in the cavity 112 can include a readily compressible fluid, such as a gaseous fluid with the same composition as ambient air.

The cover 110 can maintain a differential fluid pressure, such as a gauge pressure of the working fluid in the cavity 112, in contact with patient tissue. In an example, gauge pressure can be defined as the difference in pressure between the working fluid pressure in the cavity 112 and atmospheric pressure surrounding the cover 110.

A positive gauge pressure, such as where working fluid pressure in the cavity 112 is greater than atmospheric pressure, can create a force to stimulate patient tissue. In an example, the positive gauge pressure can create a compressive force on patient tissue exposed to the eye environment in the cavity 112, such as a compressive force proportional to the positive gauge pressure, while reducing compressive force on patient tissue in contact with the patient interface surface 119A, such as to cause the cover 110 to displace from its position against the patient tissue due to the force generated within the cavity 112.

The applied force can be related to the positive gauge pressure in the cavity 112. In an example, the applied force can include the force resulting from a positive gauge pressure, such as a range of positive gauge pressure including at least one of a range of about 5 mmHg to about 15 mmHg, a range of about 15 mmHg to about 25 mmHg, a range of about 25 mmHg to about 35 mmHg, or a range of about 35 mmHg to about 45 mmHg.

A negative (or “vacuum”) gauge pressure, such as where working fluid pressure in the cavity 112 is less than atmospheric pressure, can create an applied force on patient tissue. In an example, the negative gauge pressure can draw patient tissue into the cavity 112 to create a “pulling” force on patient tissue in the cavity 112, such as a “pulling” force proportional to the negative gauge pressure. In an example, the negative gauge pressure in the cavity 112 can cause the cover 110 to be drawn towards the patient, such as to compress patient tissue proximal to and in contact with the patient interface surface 119A.

The applied force can be related to the negative gauge pressure in the cavity 112. In an example, the applied force can include the force resulting from a negative gauge pressure, such as a range of negative gauge pressure including at least one of a range of about −5 mmHg to about −15 mmHg, a range of about −15 mmHg to about −25 mmHg, a range of about −25 mmHg to about −35 mmHg, or a range of about −35 mmHg to about −45 mmHg.

The applied force can be applied to the patient for a period of time, such as for a period of time sufficient to adjust patient perception of an indication of a headache symptom. In an example, applied force can be applied for a period, such as measured in days, weeks, months, or years.

The headache symptom can be adjusted by the apparatus 100, such as by concurrently exposing the patient eye to the composition of the working fluid in the eye environment and non-ambient pressure of the eye environment. In an example, exposing the patient eye to the working fluid composition of the eye environment can facilitate absorption of at least a part of a therapeutic fluid into the patient eye. In an example, exposing patient tissue to non-ambient pressure applied with the eye environment can apply a therapeutic force to stimulate the patient tissue.

The headache symptom and an eye condition can be affected by, such as concurrently affected by, the apparatus 100. In an example, a patient headache, such as a headache including an aura and post-aural migraine, can be accompanied by at least one of pain experienced by the patient or a decrease in ocular blood flow, such as a decrease in macular blood flow. Adjustment of the eye environment, such as by adjusting non-ambient pressure in the cavity 112, can adjust force applied to patient tissue, such as at the patient interface surface 119A, to stimulate patient tissue including a nerve proximal to the patient tissue to adjust patient perception of a headache symptom. Adjustment of the eye environment, such as by adjusting non-ambient pressure in the cavity 112, can adjust intraocular pressure (IOP), such as to adjust blood flow in the patient eye.

The apparatus 100 can be configured to adjust the eye environment, such as non-ambient pressure in the eye environment, to concurrently address at least one of the headache symptom or the eye condition. In an example, the apparatus can be configured to adjust non-ambient pressure toward at least one of a headache target cavity pressure, such as to adjust a headache symptom experienced by the patient including at least one of aura or pain, or a blood flow target cavity pressure, such as to adjust blood flow in an ocular blood vessel. In an example, the apparatus can be configured to adjust the eye environment including at least one of non-ambient pressure, such as toward at least one of a headache target cavity pressure or a target blood flow cavity pressure, or the electromagnetic environment, such as with the use of an electromagnetic (EM) energy transfer device (ETD) to generate EM energy for delivery to the patient eye, to adjust blood flow in the eye. For example, the EM ETD can generate energy in the visible light frequency range, such as pulses of energy in the visible light frequency range, to increase ocular blood flow including macular blood flow in the patient eye.

The cover 110 can include a seal 119, such as to provide a patient interface surface 119A between the cover 110 and the patient, such as to improve patient comfort when wearing the apparatus 100. The seal 119 can also serve as a barrier, such as to separate the eye environment in the cavity 112 from the surrounding environment. The seal 119 can attach to the periphery of the cover 110, such as at least a portion of the periphery of the cover 110. In an example, the seal 119 can extend continuously around the periphery of the cover, such as to form a sealing surface between the cover 110 and the patient 119 to separate the volume of the cavity 112 from the surrounding environment.

The cover 110 can include an energy transfer device (or ETD), such as a device that can transfer energy from a first object to a second object. The ETD can operate to transfer energy to the patient, such as through at least one of the cover 110 or the seal 119, such as the patient interface surface 119A. The transfer of energy can stimulate the patient tissue, such as to adjust patient perception of an indication of a headache symptom to treat, inhibit, or prevent the headache symptom. An ETD can transfer energy, such as in the form of at least one of a transfer of thermal energy, a transfer of energy through application of a force including at least one of a static force, a quasi-static force, or a dynamic force, a transfer of sonic energy, or a transfer of electromagnetic energy.

The ETD can include a temperature ETD, such as a device to affect a transfer of thermal energy through adjustment of temperature at the patient interface surface 119A. The temperature ETD can include a heating ETD, such as a device that can increase the patient interface surface 119A from a first temperature to a second temperature where the second temperature is greater than the first temperature. In an example, a heating ETD can include a resistance coil, such as a coil in communication with the control circuitry. The coil can be attached to the apparatus 100 in proximity to the seal 119 and can be capable of converting electrical power, such as from the control circuitry, into thermal energy, such as to increase the temperature of patient tissue in proximity to the cover 110 via conduction of thermal energy from the coil to the patient through the patient interface surface 119A.

The temperature ETD can include a cooling ETD, such as a device that can decrease the temperature of the patient interface surface 119A from a first temperature to a second temperature where the second temperature is less than the first temperature. In an example a cooling ETD can include at least one of a thermoelectric cooler (TEC) or a device using the Peltier effect, such as a TEC in communication with the control circuitry. The TEC can be attached to the apparatus 100 in proximity to the seal 119 and can be capable of converting electrical power, such as from the control circuitry, into a thermal sink, such as to reduce the temperature of patient tissue in proximity to the cover 110 via transfer of thermal energy from the patient to the TEC through the patient interface surface 119A.

The ETD can include a vibration ETD, such as a device to generate vibration energy for transfer to the patient. The vibration ETD can include a rotating unbalance device, such as a rotating mass where the center of mass of the vibration ETD does not align with the center of rotation of the vibration ETD. In an example, the vibration ETD can include an electric motor with an eccentric rotating mass to generate vibrational energy. The vibration ETD can be in communication with the cover 110, such as to transmit vibrational energy from the vibration ETD to the patient through the patient interface surface 119A. Transmission of vibration at different frequencies can be affected by adjusting the speed of the electric motor. In an example, the vibration ETD can include a piezoelectric element, such as a piezo uni-morph or piezo bi-morph in communication with the cover 110, to transmit vibration energy from the piezoelectric element to the patient through the patient interface surface 119A.

The ETD can include an acupuncture ETD, such as a device to locate and insert a needle into patient tissue. The acupuncture ETD can include the cover 110, such as the needle can be located on the patient interface surface 119A and oriented perpendicularly to the same, such as to penetrate the patient tissue when the patient interface surface 119A is brought into contract with the patient. Penetration of the needle into patient tissue can be controlled, such as by adjusting non-ambient pressure in the cavity 112 to vary force between the cover 110 and patient tissue at the patient interface surface 119A. In an example, negative cavity pressure can draw the cover 110 and patient interface surface 119A closer to the patient tissue, such as to cause the needle to penetrate the patient tissue. The depth of patient tissue penetration can be based on the level of negative cavity pressure applied to the cavity 112.

The needle can be in communication with another ETD, such as at least one of the temperature ETD, the vibration ETD, or an electrostimulation ETD to enhance an effect of the acupuncture ETD. The needle can include a dissolvable needle, such as a needle constructed from a therapeutic substance and configured to be inserted into patient tissue and subdermally absorbed into patient tissue, such as to treat, inhibit, or prevent an indication of a headache symptom.

Acupressure (or shiatsu) includes a form of therapy involving the application of pressure to patient tissue. In an example, acupressure can be applied to a patient to adjust patient perception of pain, such as to relieve patient pain. An acupressure pressure point can be defined as an area on the human body to which pressure can be applied, such as to adjust patient perception of pain.

The ETD can include an acupressure ETD to apply a localized pressure to an area of patient tissue, such as to generate a localized force on patient tissue. The acupressure ETD can be configured to apply localized force to an acupressure point such as concurrently to one or more acupressure points. An acupressure ETD can include at least one of a protuberance 191, such as a projection or “bump” protruding from the patient interface surface 119A, or a recess 192, such as a depression or “hole” extending into the patient interface surface 119A. In an example, the acupressure ETD can be applied to patient tissue while adjusting the eye environment, such as non-ambient pressure in the eye environment, to vary acupressure force applied to patient tissue.

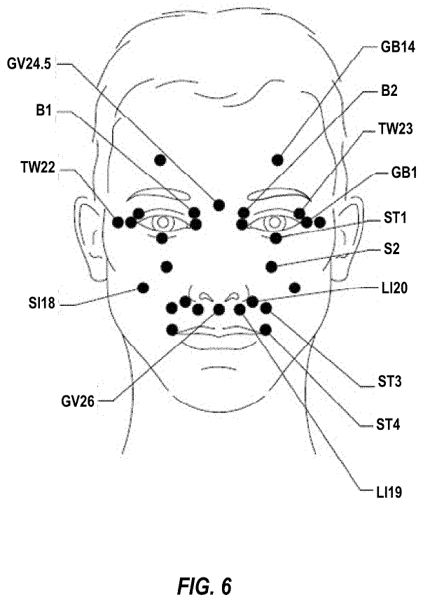

FIG. 6 shows an illustration of acupressure points on facial tissue. Facial acupressure pressure points can include at least one of Seal Place (GV24.5), Eyes Bright (B1), Harmony Bone (TW22), Yang White (GB14), Drilling Bamboo (B2), Silk Bamboo Lollow (TW23), Hearing Meeting (GB1), Receive Tears (ST1), Four Whites (S2), Welcome Perfume (LI20), Cheekbone (SI18), Middle of Person (GV26), Facial Beauty (ST3), Earth Granary (ST4), or Grain Bone (LI19). The facial acupressure pressure points can by symmetrical, such as bilaterally symmetrical about a centerline of the face, such as a centerline including at least one of a centerline extending vertically between the eyes and the nostrils of the face or a centerline connecting to acupressure points, such as the labeled points GV24.5 and GV26. In an example, the acupressure ETD can be configured to locate the protuberance 191 over a facial acupressure point and contact pressure applied to the facial acupressure point, such as by adjustment of non-ambient pressure in the cavity 112, to stimulate patient tissue.

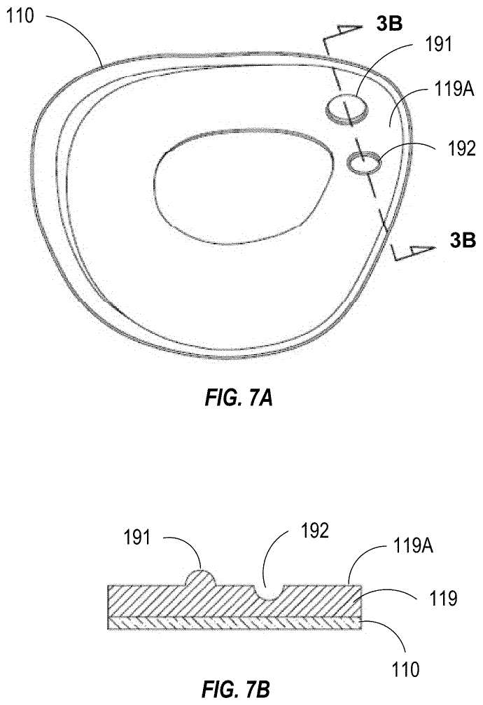

FIGS. 7A and 7B show an example of a protuberance 191 and a recess 192, such as located on the patient interface surface 119A. The protuberance 191 can include a feature, such as one or more features, that can be adjusted, such as to improve effectiveness of the acupressure ETD. The protuberance 191 can assume a generally circular shape, such as shown in FIG. 7A, or any non-circular shape. The protuberance 191 can assume a generally biaxially symmetric cross section, such as shown in FIG. 7B, or any non-symmetric cross section. The height of the protuberance 191 can vary the level of force applied to the patient tissue. The height of the protuberance 191 can be defined as the distance from a reference surface, such as the patient interface surface 119A, to the maximum excursion of the protuberance 191 from the reference surface. In an example, the protuberance height can include a range of height, such as at least one of a range from about 0 mm to about 2 mm, a range from about 2 mm to about 5 mm, a range from about 5 mm to about 7.5 mm, or a range of greater than about 7.5 mm. The protuberance 191 can include a needle, such as an acupuncture needle.

The recess 192 can include a feature, such as one or more features, that can be adjusted, such as to improve effectiveness of the acupressure ETD. The recess 192 can assume a generally circular shape, such as shown in FIG. 7A, or any non-circular shape. The recess 192 can assume a generally biaxially symmetric cross section, such as shown in FIG. 7B, or any non-symmetric cross section. The depth of the recess 192 can vary the level of force applied to the patient tissue. The depth of the recess 192 can be defined as the distance from a reference surface, such as the patient interface surface 119A, to the maximum excursion of the recess 192 from the reference surface. In an example, the recess 192 depth can include a range of depth, such as at least one of a range from about 0 mm to about 2 mm, a range from about 2 mm to about 5 mm, a range from about 5 mm to about 7.5 mm, or a range of greater than about 7.5 mm.

The acupressure ETD can include a portion formed as an integral part of the patient interface surface 119A. In an example, the protuberance 191 or the recess 192 can be incorporated as a feature into the mold or die used to form the patient interface surface 119A.

The acupressure ETD can include a component separate from the patient interface surface 119A. In an example, the protuberance 191 can include a molded protuberance component that can adhere to the patient interface surface 119A, such as a molded protuberance component that can be bonded to a location on the patient interface surface 119A to precisely position the protuberance component with respect to an acupressure pressure point on the patient tissue. In an example, the protuberance 191 can in include a molded protuberance component that can be located over the acupressure pressure point on the patient tissue and then bonded to the patient interface surface 119A upon locating the cover 110 over the eye of the patient.

The acupressure ETD can include a flange cover, such as a flange cover configured overlay the patient interface surface 119A. A flange cover can include any structure that can be located between the patient interface surface 119A and the patient. The flange cover can include a base structure, such as a sheet-like material with an adhesive on one or both sides of the sheet-like material, to overlay at least a portion of the patient interface surface 119A, and a protuberance structure including a protuberance 191, connected to the base structure. In an example, the flange cover can be configured to overlay the patient interface surface 119A, such as to position the protuberance for contact with the patient tissue when the cover 110 is located against the patient. In an example, the flange cover can be adjusted with respect to the patient interface surface 119A, such as to relocate the protuberance structure from a first position to a second position different from the first position to better align the protuberance structure with a location on the patient tissue, such as an acupressure pressure point on the patient.

The ETD can include a sonic ETD, such as a device attached to or in proximity of the cover 110, to generate energy in the sonic frequency range for transmission to the patient to stimulate patient tissue. The sonic frequency range can include a frequency range from about 2 Hertz (Hz) to about 20 kilohertz (kHz).

The ETD can include an electromagnetic (or EM) ETD, such as a device attached to or in proximity of the cover 110, to generate energy in the EM frequency range for transmission to the patient to stimulate patient tissue. The EM frequency range can include a radio frequency range including a frequency range from about 20 kHz to about 300 megahertz (MHz). The EM frequency range can include a microwave frequency range including a frequency range from about 300 MHz to about 300 gigahertz (GHz). The EM frequency range can include an infrared frequency range including a frequency range from about 300 GHz to about 430 tetrahertz (THz). The EM frequency range can include a visible light frequency range including a frequency range from about 430 THz to about 750 THz. The EM frequency range can include an ultraviolet frequency range including a frequency range from about 750 THz to about 3 petahertz (PHz).

In an example, an EM ETD can include a visible light ETD, such as a device capable of transmitting radiation in the visible light frequency range from the visible light ETD into the patient eye, such as to stimulate ocular patient tissue including retinal tissue to increase ocular blood flow including macular blood flow. The visible light ETD can generate pulses of visible light (or pulsed light), such as to stimulate the ocular tissue. In an example, the frequency of the pulsed light can vary in a range, such as in a frequency range of at least one of about 0.25 Hz to about 500 Hz, about 2 Hz to about 100 Hz, about 10 Hz to about 70 Hz, or from about 20 Hz to about 50 Hz.

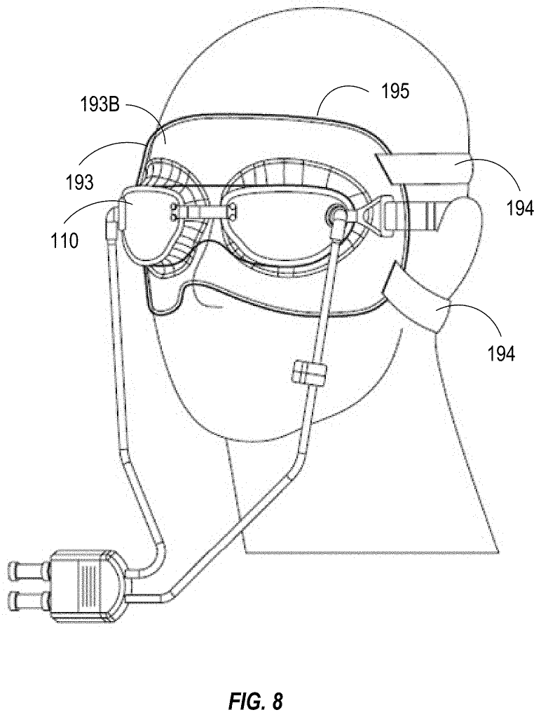

FIG. 8 shows an example of the cover 110 including an anterior plate 193. The anterior plate 193 can include an extension of the cover 110 configured to cover at least a portion of patient tissue proximal to an anterior portion of the skull. The anterior portion of the skull can include a bone of the skull such as at least a portion of one of a frontal bone, a parietal bone, a temporal bone, a sphenoid bone, an ethmoid bone, a nasal bone, a lacrimal bone, a zygomatic bone, a maxilla bone, or a mandible bone.

The anterior plate 193 can be coupled to and work in combination with the cover 110, such as to adjust patient perception of an indication of a headache symptom to treat, inhibit, or prevent the headache symptom. In an example, the anterior plate 193 can be affixed to the cover 110, such as the anterior plate 193 and the cover 110 can form a single component of the apparatus 100. In an example, the anterior plate 193 can be removably secured to the cover 110, such as the anterior plate 193 and the cover 110 can be attached to each other to form a single component of the apparatus 100 or removably detached one from the other to form two components.

The distance between the anterior plate 193 and the patient tissue can be adjusted, such as by adjusting non-ambient pressure in the cavity 112. In an example, increasing the level of vacuum (e.g., increasing applied negative pressure) or decreasing the level of positive pressure in the cavity 112 can cause the anterior plate 193 to be drawn closer to the patient tissue, such as to decrease the distance between the anterior plate 193 and the patient tissue. For example, the level of vacuum can be increased such as to cause the anterior plate 193 to contact the patient tissue. In an example, decreasing the level of vacuum (e.g., decreasing applied negative pressure) or increasing applied positive pressure in the cavity 112 can cause the anterior place 193 to be located further from the patient tissue, such as to increase the distance between the anterior plate 193 and the patient tissue.

The anterior plate 193 can include an anterior plate harness 194, such as a device capable of locating and retaining the anterior plate 193 over the patient tissue. The anterior plate harness 194 can include a tether, such as a retention device including a strap, attached to the anterior plate 193 and configured to locate the anterior plate 193 on the patient over the patient tissue.

The anterior plate harness 194 can include a tensioner, in communication with the tether, configured to generate tension in the tether, such as to draw the anterior plate 193 into contact with the patient tissue. The tensioner can include any device configured to create or retain tension in a tether or similar tether-like device, such as at least one of a winch mechanism, a belt tensioning device, a drawstring-style device, a come-along device, or an adjustable coupler, such as a turnbuckle. The tensioner can include a sensor 130, such as a tension sensor to sense tension force in the tether. The tensioner can be manually adjusted, such as a user can manipulate the tensioner to increase or decrease tension in the tether. The tensioner can be remotely adjusted, such as with a tensioner actuator connected to the tensioner to increase or decrease tension in the tether. The tensioner actuator can include any force-generating device, such as a pneumatic actuator, a hydraulic actuator, or an electric actuator including an electric motor. The tensioner actuator can be in communication with the control circuitry 140, such as to adjust tension in the tether by adjusting the tensioner actuator based on a tension feedback control circuit.

The anterior plate 193 can contact at least a portion of patient tissue proximal to the anterior portion of the skull, such as to stimulate the patient tissue. Patient tissue proximal to the anterior portion of the skull can include tissue covering the skull, such as including epidermis, dermis, hypodermis, and one or more structures residing within the tissue, such as including a blood vessel and a cranial nerve. The cranial nerve can include any nerve located in proximity to the anterior portion of the skull, such as at least one of a trigeminal nerve or a facial nerve. Patient tissue proximate to the anterior portion of the skull can include tissue in proximity to at least a portion of the interior of the skull, such as the meninges including a portion of dura mater, arachnoid mater, or pia mater, and other tissues associated with the meninges, such as including an interior blood vessel or interior cranial nerve.

In an example, the tether can encircle at least a portion of the patient head to locate the anterior plate 193 over the patient tissue and the tensioner can be adjusted, such as to cause the anterior plate 193 to contact the patient tissue. In bringing the anterior plate 193 into contact with patient tissue, the tensioner can be further adjusted, such as to generate plate contact pressure between the anterior plate 193 and the patient tissue. The magnitude of the plate contact pressure can be adjusted, such as by at least one of adjusting tension in the tether with the tensioner or adjusting non-ambient pressure in the cavity 112.

The plate contact pressure generated by the anterior plate 193 against the patient tissue can stimulate the patient tissue to adjust a perceived patient headache symptom, such as to treat, inhibit, prevent the headache symptom. The effect of the plate contact pressure to adjust the perceived patient headache symptom can be enhanced through additional stimulation of the patient tissue, such as with an energy transfer device (ETD).

The anterior plate 193 can include an ETD as described elsewhere in this application to stimulate patient tissue. The ETD can include a protuberance 191, such as located between the anterior plate 193 and patient tissue. In an example, the protuberance 191 can be positioned over and brought into contact with a facial acupressure point, such as to apply force to patient tissue. The applied force can be varied, such as by adjusting at least one of the non-ambient pressure in the eye environment or the tensioner. In an example, the ETD can attach to and removably detach from the anterior plate 193.

The anterior plate 193 can be constructed from a sheet material, such as a material formed in a sheet. The sheet material can include a rigid sheet material, such as at least one of a thermoset material or a thermoplastic material. The rigid sheet material can be conformed to the patient tissue, such as conformed to facial contours of the patient. In an example, the rigid thermoplastic sheet material can conform to the patient tissue, such as facial contours of the patient covered by facial tissue, by heating the sheet to a temperature above a glass transition temperature of the sheet material and subsequently forming the sheet to the facial contours of the patient. The anterior plate 193 can include a first anterior plate surface 193A (not shown), such as a surface of the anterior plate facing the patient tissue, and a second anterior plate surface 193B, such as a surface opposite the first anterior plate surface 193A.

An anterior plate gasket 195 can be located around the periphery of the anterior plate 193, such as to create an anterior plate cavity between the first anterior plate surface 193A (not shown) and the patient tissue. The anterior plate gasket 195 can create an air-tight interface between the first anterior plate surface 193A (not shown) and patient tissue proximate to the anterior portion of the skull, such as to support a gauge pressure in the anterior plate cavity, such as an anterior plate cavity pressure. Application of at least one of a positive gauge pressure or a negative gauge pressure, such as with the pressure source 150, can generate a force on the patient tissue exposed to the gauge pressure in the anterior plate cavity, such as to stimulate the patient tissue to adjust an indication of a headache symptom to treat, inhibit, or prevent the headache symptom.

The sheet material can include a flexible sheet material, such as at least one of an elastomer or a polymer including high-density polyethylene. The flexible sheet material can conform to patient tissue, such as facial contours of the patient tissue, and can adhere to the patient tissue, such as due to at least one of the nature of flexible sheet material, processing of the flexible sheet material, or by locating an adhesive between the flexible sheet material and the patient tissue. The flexible sheet material can incorporate an ETD, such as to locate and retain the ETD against the anterior patient tissue. In an example, the ETD can be embedded in the flexible sheet material for placement against the anterior patient tissue or the ETD can be placed against the anterior patient tissue and covered by the flexible sheet material, such as to locate and retain the ETD against the anterior patient tissue.

A flex sheet cavity can be formed between the flexible sheet material and the patient tissue. The flex sheet cavity can be formed by adhering at least a portion of the flexible sheet material to the patient tissue and preventing the remaining portion of the flexible sheet material from adhering to the patient tissue. In an example, the periphery of the flexible sheet material can be adhered to the patient tissue and the flexible sheet material not forming the periphery can be prevented from adhering to the patient tissue, such as to form a “pouch” or flex sheet cavity between the flexible sheet material and the patient tissue.

The flex sheet cavity can be in communication with the pressure source 150 and configured to retain gauge pressure against the patient tissue, such as to generate a force due to gauge pressure on the patient tissue exposed to the gauge pressure and stimulate the patient tissue. In an example, an inflatable pillow in communication with the pressure source 150 and configured to retain gauge pressure can be located in the flex sheet cavity, such as to generate a force reacted by the patient tissue to stimulate the patient tissue.

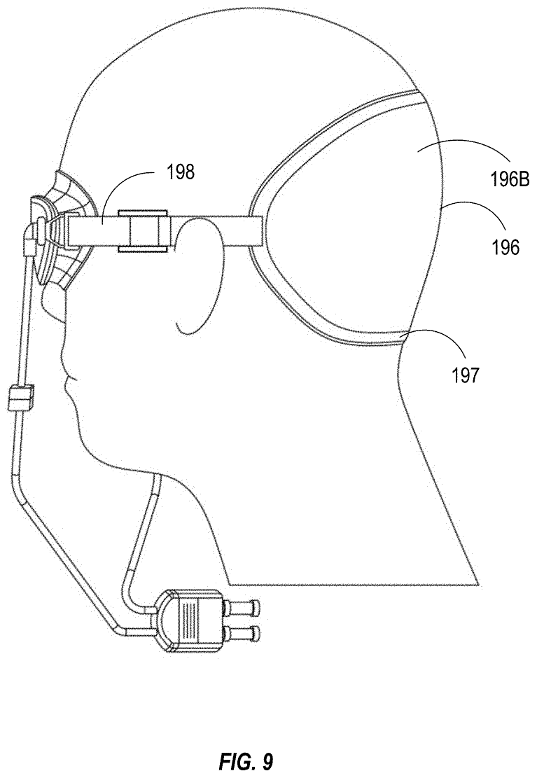

FIG. 9 shows an example of the cover 110 including a posterior plate 196. The cover 110 can include the posterior plate 196, such as a component configured to cover at least a portion of patient tissue proximal to a posterior portion of the skull. The posterior portion of the skull can include a bone of the skull such as at least a portion of at least one of a parietal bone, a frontal bone, an occipital bone, or a temporal bone.

The posterior plate 196 can contact at least a portion of patient tissue proximal to the posterior portion of the skull, such as to stimulate the patient tissue. Patient tissue proximate to the posterior portion of the skull can include tissue covering the skull including epidermis, dermis, hypodermis, and one or more structures residing within the tissue including a blood vessel and a cranial nerve. The cranial nerve can include any nerve located in proximity to the posterior portion of the skull, such as an occipital nerve. Patient tissue proximate to the posterior portion of the skull can include tissue in proximity to at least a portion of the interior of the skull, such as the meninges including a portion of dura mater, arachnoid mater, or pia mater, and other tissues associated with the meninges including an interior blood vessel or interior cranial nerve.

The posterior plate 196 can be coupled to and work with the cover 110, such as with a posterior plate tether 198, to adjust patient perception of an indication of a headache symptom to treat, inhibit, such as a device capable of locating and retaining the posterior plate 196 in contact with the patient tissue.

The posterior plate tether 198 can include at least one of a rigid posterior plate tether or a flexible posterior plate tether. The rigid posterior plate tether can include adjustable headgear, such as including an adjustable harness, strip ratchet headband, or similar devices to locate and secure the posterior plate 196 to the patient and can include a tensioner configured to draw the posterior plate into contact with the patient tissue. The flexible posterior plate tether can include a strap, such as to locate the posterior plate 196 against the patient, and a tensioner, such as to draw the posterior plate 196 into contact with the patient.

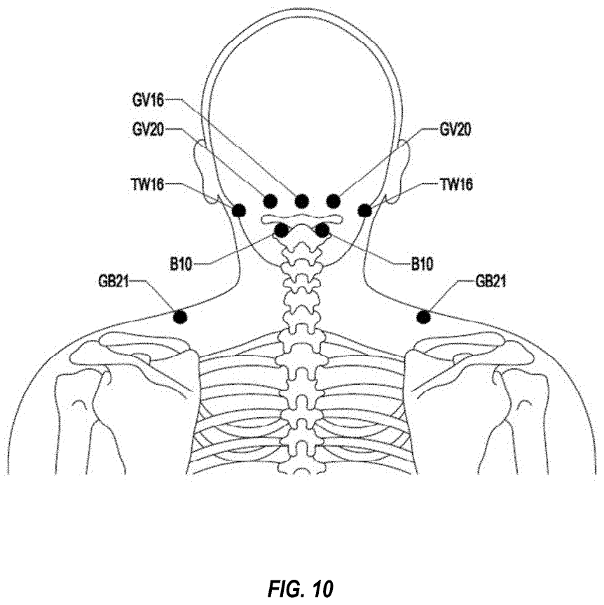

FIG. 10 shows an illustration of posterior acupressure points on a patient. Acupressure pressure points can include at least one of Wind Mansion (GV16), Crown Chakra (GV20), Window of Heaven (TW16), Heavenly Pillar (B10), or Shoulder Well (GB21). In an example, a protuberance 191 can be located on the first posterior plate surface 196A (not shown) over a posterior acupressure point and contact pressure applied to the posterior acupressure point, such as by adjustment of tension in the posterior plate tether 198, to stimulate patient tissue.

The posterior plate 196 can include an ETD that can operate to transfer energy to the patient, such as through contact with the patient tissue, such as to stimulate a posterior acupressure point. An ETD can include at least one of a temperature ETD, a vibration ETD, an acupuncture ETD, an electrostimulation ETD, an acupressure ETD, a sonic ETD, or an EM ETD.

In an example, the ETD can include a protuberance 191, such as located between the posterior plate 196 and patient tissue. In an example, the protuberance 191 can be positioned over and brought into contact with a posterior acupressure point to apply force to patient tissue. The applied force can be varied, such as by adjusting at least one of the non-ambient pressure in the eye environment or the tensioner. In an example, the ETD can attach to and removably detach from the posterior plate 193.

The posterior plate 196 can be constructed from a sheet material. The sheet material can include a rigid sheet material, such as at least one of a thermoset material or a thermoplastic material. The rigid sheet material can be conformed to the patient tissue, such as conformed to posterior cranial contours of the patient. In an example, the rigid thermoplastic sheet material can conform to the patient tissue by heating the sheet to a temperature above a glass transition temperature of the material and subsequently forming the sheet to the posterior cranial contours of the patient. The posterior plate 196 can include a first posterior plate surface 196A (not shown), such as a surface of the posterior plate facing the patient tissue, and a second posterior plate surface 196B, such as a surface opposite the first posterior plate surface 196A (not shown).

Referring again to FIG. 9, a posterior plate gasket 197 can be located around the periphery of the posterior plate 196, such as to create a posterior plate cavity between the first posterior plate surface 196A (not shown) and the patient tissue. The posterior plate gasket 197 can create an air-tight interface between the first posterior plate surface 196A (not shown) and patient tissue proximate to the posterior portion of the skull, such as to support a posterior plate cavity pressure in the posterior plate cavity. Application of at least one of a positive gauge pressure or a negative gauge pressure, such as with the pressure source 150, can generate a force on the patient tissue exposed to the gauge pressure, such as to stimulate the patient tissue to adjust an indication of an indication of a headache symptom including to treat, inhibit, or prevent the headache symptom.

The sheet material can include a flexible sheet material, such as at least one of an elastomer or a polymer including a high-density polyethylene. The flexible sheet material can conform to patient tissue, such as posterior cranial contours of the patient tissue, and can adhere to the patient tissue, such as due to the nature of flexible sheet material, processing of the flexible sheet material, or by locating an adhesive between the flexible sheet material and the patient tissue. The flexible sheet material can incorporate an ETD, such as to locate and retain the ETD against the posterior patient tissue. In an example, the ETD can be embedded in the flexible sheet material for placement against the posterior patient tissue or the ETD can be placed against the posterior patient tissue and covered by the flexible sheet material.

The flexible sheet material can form a flex sheet cavity, such as described elsewhere in this application, between the flex sheet material and patient tissue proximal to the posterior portion of the skull. An inflatable pillow, such as described elsewhere in this application, can be located in the flex sheet cavity, such as to generate a force reacted by the patient tissue to stimulate the patient tissue. In an example, the inflatable pillow can be in communication with the pressure source 150.

Referring again to FIG. 1, the fluid regulator 120 can regulate the flow of fluid between two reservoirs, such as the fluid flow between the cavity 112 and a fluid source 170, such as a pressurized gas cylinder. The fluid regulator 120 can include a regulator valve, such as to regulate flow rates between the first and second reservoirs. The regulator valve can include a passive valve, such as a check valve that closes as pressure exceeds a critical value. In an example, a fluid regulator 120 with a check valve can be located between the cover 110 and a fluid source 170, such as if the pressure of the fluid source 170 exceeds a critical value, such as a pressure that can cause damage to a patient eye, the check valve can close to isolate pressure of the fluid source 170 from the patient eye, such as to protect the patient eye from excessive force. The regulator valve can include an active valve, such as an electrically-modulated valve including a servo valve, or a proportional valve, such as a piezo-actuated proportional valve. In an example, the regulator valve can receive a control signal, such as from the control circuitry 140, to modulate the position of the electrically-modulated spool with respect to the valve body, such as to regulate fluid flow through the electrically-modulated valve.

The fluid regulator 120 can attach to a fluid source 170, such as to regulate the flow of fluid from the fluid source 170 to the cavity 112. The fluid source 170 can include a fluid vessel, such as a storage container of pressurized gaseous fluid. The fluid source 170 can include a generator device, such as a device that concentrates or distills a constituent fluid from another fluid. In an example, a generator device can include a concentrator, such as an oxygen concentrator or a carbon dioxide concentrator. In an example, a generator device can include an atomizer, such as an ultrasonic humidifier or an aerosolizer, to transform a liquid therapeutic fluid, such as a miscible solution or colloidal suspension, into a gaseous working fluid, such as a therapeutic mist or fog.

The fluid regulator 120 can communicate with apparatus 100, such as the fluid regulator 120 can communicate with the cavity 112. In an example, the fluid regulator 120 can be connected to the cover 110, such as with the conduit 117 in direct communication with the cover 110 through the second port 116. In an example, the fluid regulator 120 can be connected to the conduit 117 in communication with the cover 110 by a tube connector 118, such as a Y-connector. In an example, the fluid regulator 120 can be connected to the control circuitry 140, such as to receive a control signal from the control circuitry 140 to adjust the position of a servo valve.

The sensor 130 can sense an indication of the eye environment in the cavity 112, such as at least one of an indication of a characteristic of the working fluid in the cavity 112 or an indication of a physiological parameter of the patient. The sensor 130 can include sensor circuitry, such as sensor circuitry to receive an indication of a physical parameter sensed by the sensor 130 and process the received indication, such as into an indication including an electrical signal suitable to be received by at least one of the control circuitry 140 or the pressure source 150.

The sensor 130 can be located in proximity to the apparatus 100, such as in communication with the cavity 112 or at least partially attached to the patient. In an example, the sensor 130 can be separate from the apparatus 100. For example, the sensor 130 can include a handheld pressure gauge, such as to be pressed against a measurement septum located over the port 114 to sense an indication of working fluid pressure in the cavity 112. In an example, the sensor 130 can be in fluidic communication with the cavity 112, such as the sensor 130 can be located in the cavity 112 or on the control circuitry 140 in fluidic communication with the cavity 112. In an example, the sensor 130 can be at least partially attached to the patient, such as to a surface of the eye including an anterior surface of the eye or patient tissue covering the skull including tissue over the frontal, parietal, sphenoid, temporal, zygomatic, maxillary, occipital, and mandibular bones. For example, the sensor 130 can include an electroretinography device, such as part of which can include an electrode attached to patient tissue to sense an indication of electrical activity in the patient including electrical activity associated with a pattern electroretinography (or PERG) test.

The sensor 130 can be in electrical communication with the apparatus, such as at least one of the control circuitry 140 or the pressure source 150. The sensor 130 can provide at least one of continuous or periodic (e.g. intermittent) sensing of the working fluid, such as for monitoring an indication of the eye environment with the sensor 130, or an indication of the physiological parameter associated with the patient, such as IOP or CSFP.

The sensor 130 can include an IOP sensor, such as a device to sense an indication of an intraocular pressure (TOP) level in the eye. The IOP sensor can include at least one of an invasive IOP sensor, such as an IOP sensor implantable in an intraocular space of the eye to sense IOP including a sensor from Implandata Ophthalmic Products GmbH (Hannover, Germany) offered for sale under the trademark EYEMATE® or a non-invasive IOP sensor, such as an IOP sensor to sense IOP without implantation into the body including a contact lens-based sensor from Sensimed AG (Lausanne, Switzerland) offered for sale under the trademark SENSIMED TRIGGERFISH®.

The IOP sensor can include at least one of a continuous IOP sensor, such as an IOP sensor capable of continuous sensing of IOP level in the patient eye, or a periodic IOP sensor, such as an IOP sensor that capable of sensing IOP level in the patient eye at periodic or aperiodic intervals. In an example, the periodic IOP sensor can include a tonometer, such as a handheld tonometer designed for patient self-monitoring of IOP. The data sensed by the IOP sensor can be received by the control circuitry 140, such as to facilitate use of the apparatus 100.

The sensor 130 can include a cardiac sensor, such as to detect an indication of cardiac activity in a patient. An indication of cardiac activity can include at least one of an indication of systemic blood pressure, such as an indication of systolic and an indication of diastolic blood pressure, or an indication of heart rate.

The cardiac sensor can include a blood pressure (BP) sensor, such as a device to sense an indication of blood pressure level including systemic blood pressure level, in the patient. The BP sensor can include at least one of an invasive BP sensor, such as a BP sensor implantable within the patient, and a non-invasive BP sensor, such as a BP sensor that can sense BP without implantation within the patient body.

The sensor 130 can include a working fluid flow sensor, such as a device to sense an indication of working fluid flow including at least one of volumetric flow rate or mass flow rate into or out of the cavity 112. The sensor 130 can include a humidity sensor, such as a device to sense an indication of the relative humidity of the working fluid in the cavity 112. The sensor 130 can include a thermometer, such as a device to sense an indication of the temperature of the working fluid in the cavity 112. The sensor 130 can include a displacement sensor, such as a device to sense an indication of displacement including an optical coherence tomography device configured to sense displacement of structures associated with the patient eye.

The sensor 130 can include a pressure sensor, such as a device to sense an indication of working fluid pressure in the cavity 112. The pressure sensor can be located in proximity to the cavity 112, such as in communication with the cavity 112. In an example, the pressure sensor can include a cavity pressure sensor, such as a pressure sensor located in the cavity 112.

Static cavity pressure level in the cavity 112, such as the pressure level sensed by the pressure sensor when the pressure source 150 is not adjusting working fluid pressure in the cavity 112, can be the same at any location in the cavity 112. Dynamic cavity pressure level, such as the pressure level sensed by the pressure sensor when the pressure source 150 is adjusting working fluid pressure in the cavity 112, can vary depending on the location of the pressure sensor in communication with the cavity 112.

The sensor 130 can include a pressure sensor in combination with another indication, such as an indication of the operating state of the pressure source 150, to estimate a static cavity pressure level in the cavity 112. In an example, the pressure sensor, such as a pressure-flow sensor including a sensor that can measure both working fluid pressure (static and dynamic) and working fluid flow at a measurement location, can be located in proximity to the pressure source 150, such as an inlet port or an outlet port of the pressure source 150, to sense an indication of dynamic pressure at the pressure sensor location and include circuitry, such as sensor circuitry to receive an indication of the operation state of the pressure source 150 including an indication of flow rate (e.g., pump speed can be proportional to flow rate). The pressure-flow sensor can process at least one of the indication of dynamic pressure or the indication of flow rate, such as to form a control signal that can be received by the pressure source 150 to achieve a static cavity pressure level, such as a target pressure level, in the cavity 112. The control signal can be based on a relationship between the indication of dynamic pressure and the indication flow rate, such as a relationship between pressure and flow including the relationship described by a p-Q (e.g., pressure-flow) chart that can account for the operating characteristics of the pressure source 150.

In an example, the pressure sensor can be located in proximity to the pressure source 150. The control circuitry 140 can be configured to receive an indication of dynamic pressure from the pressure sensor and an indication of the operation state of the pressure source 150 including an indication of pump speed. The control circuitry 140 can process at least one of the indication of dynamic pressure or the indication of pressure source 150 operation state, such as to form a control signal that can be received by the pressure source 150 to achieve a static cavity pressure level, such as a target pressure level, in the cavity 112.

The sensor 130 can include a concentration sensor or a working fluid composition sensor, such as a device to sense an indication of a chemical constituent in the working fluid. In an example, the concentration sensor can be configured to sense an indication of the working fluid, such as a constituent in the working fluid. The constituent in the working fluid, such as the constituent in the working fluid delivered to the cavity 112, can include a therapeutic fluid. In at least one example, the working fluid composition sensor can sense a therapeutic fluid, such as at least one of (CO₂), oxygen (O₂), nitric oxide (NO), ozone (O₃), nitrogen, helium (He), hydrocarbons including fluorocarbons and perfluorocarbons, sulfur hexafluoride, cannabinoids including tetrahydrocannabinol (THC) and cannabidiol (CBD), or a combination of therapeutic gases.

The sensor 130 can include a biomarker sensor, such as a device to sense an indication of a biomarker including a chemical constituent. A chemical constituent in the working fluid can include a biomarker, such as a biomarker emitted by the patient eye or sensed within the patient eye. A biomarker can suggest a physiological state of the eye, such as a state of distress where medical intervention can be required. The biomarker sensor can include a ketone, such as can be detected with a volatile gas sensor including a quartz crystal nanobalance (QCN) sensor, glucose, such as can be detected with an optical glucose sensor including an OCT imaging system, oxygen levels, such as can be detected with a non-invasive optical oxygen sensor, dissolved salts, such as can be detected with a salinity sensor, and vascular endothelial growth factor (or VEGF), such as can be detected with an aptamer-based sensor including the sensor and methods described in the publication “Flexible FET-Type VEGF Aptasensor Based on Nitrogen-Doped Graphene Converted from Conducting Polymer”, by Kwon, et at., ACS Nano, Vol. 6, #2, pages 1486-1493, published February 2012, and incorporated herein by reference in its entirety. A biomarker can include at least one of an enzyme, such as matrix metallopeptidase 9 (MPP-9), that can be detected with an enzyme sensor or a protein, such as brain-derived neurotrophic factor (BDNF), that can be detected with a protein sensor.

The sensor 130 can include a biosensor, such as a sensor configured to sense an indication of a physiological parameter associated with a patient. A physiological parameter can include an indication of a physiological process associated with the patient, such as a process associated with a patient eye or process associated with physiological activity of the patient eye. In an example, a physiological parameter can include at least one of an indication of intraocular pressure (TOP) in the patient eye, such as an TOP level, an indication of cerebrospinal fluid pressure (CSFP) associated with the patient, such as a CSFP level, an indication of cardiac activity, such as at least one of systemic blood pressure or heart rate. A physiological parameter can include an indication of retinal activity, such as measured by an electroretinography device including a pattern electroretinography (or PERG) device.

The sensor 130 can include an imaging sensor to sense an indication of the eye, such as an intraocular portion of the eye. The imaging sensor can be located in proximity to the eye, such as attached to apparatus 100 including the cover 110 or exist separately from the apparatus including as a stand-alone device. In an example, the imaging sensor can include a camera, such as a single image capture camera or a multi-image capture camera including a video camera, such as the one or more captured images can be transferred to the apparatus 100 for image processing. In an example, the imaging sensor can include an optical coherence tomography (OCT) device.

The sensor 130 can include a blood flow sensor, such as an ocular blood flow sensor. The blood flow sensor can include an invasive blood flow sensor ocular imaging system, such as a blood flow sensor and imaging system that requires at least a part component of the system to be inserted into the patient. In an example, an invasive blood flow sensor an invasive ocular imaging system can include a fluorescein angiography system.

The blood flow sensor can include a non-invasive ocular blood flow sensor, such as a blood flow sensor that does not require insertion into the patient. The non-invasive ocular blood flow sensor ocular imaging system can include a system to sense an indication of ocular blood flow from a patient or circuitry to process information from the patient to yield an indication of ocular blood flow. An indication of ocular blood flow can include at least one of peak systolic blood velocity (PSV), end diastolic blood velocity (EDV), mean blood velocity (MV), resistivity index (RI), such as RI=(PSV−EDV)/PSV, or pulsatility index (PI), such as PI=(PSV−EDV)/MV.

The non-invasive ocular blood flow sensor ocular imaging system can include an ocular energy source, such as to radiate illuminate a tissue including ocular tissue with energy to elicit a response from the tissue that can be sensed with a sensor. The ocular tissue can be illuminated with electromagnetic (EM) energy generated by the ocular energy source, such as EM energy in a frequency range from about 3 hertz (Hz) to about 300 exahertz (EHz). In an example, an ocular energy source can include a diffuse light source, such as generated by a light bulb, and a collimated light source, such as generated by a laser diode.

The non-invasive ocular blood flow sensor ocular imaging system can include an ocular blood flow sensor, such as to sense energy radiated from ocular tissue including energy elicited from the ocular tissue by illuminating the ocular tissue with an energy source. An ocular blood flow sensor can be configured to sense EM energy, such as EM energy in a frequency range from about 3 hertz (Hz) to about 300 exahertz (EHz).

In an example, the ocular blood flow sensor can include an ultrasonic sensor, such as an ultrasonic sensor configured to sense EM energy in a frequency range from about 3 Hz to about 300 gigahertz (GHz) including a frequency range from about 20 kilohertz (kHz) to about 400 kHz and a frequency range of about 1 megahertz (MHz) to about 18 MHz.

The ocular blood flow sensor can include a charge coupled device (CCD) sensor such as including a complementary metal-oxide-semiconductor (CMOS) sensor. The CCD sensor can be configured to sense EM energy in a frequency range, such as at least one of a range from about 300 GHz to about 300 exahertz or EHz including a frequency range from about 300 GHz to about 400 tetrahertz or THz (infrared radiation, corresponding to wavelengths of about 1,000 micrometers to about 750 nanometers or nm), a frequency range from about 400 THz to about 800 THz (visible light, corresponding to wavelengths of about 750 nm to about 375 nm), and a frequency range from about 800 THz to about 30 petahertz or PHZ (ultraviolet radiation, corresponding to wavelengths of about 375 nm to about 10 nm).

The non-invasive ocular blood flow sensor ocular imaging system can include a color doppler imaging (CDI) system, such as a medical ultrasonic imaging system with at least one of an ocular energy source, such as an ultrasonic transducer, an ocular blood flow sensor, such as an ultrasonic receiver, or a combination of ocular energy source and ocular blood flow sensor, such as an ultrasonic transceiver. In an example, the CDI system can be configured with an energy source capable of generating EM energy at a frequency of about 6.5 MHz.

The non-invasive ocular blood flow sensor system ocular imaging can include a laser speckle flowgraphy (LSF) or laser speckle contrast imaging (LSCI) system. In an example, the LSF system can be configured with an energy source capable of generating EM energy at a frequency of about 361 THz (corresponding to a wavelength of about 830 nm). In an example, the LSF system can include a system from Nidek Co., Ltd. (Aichi, Japan) offered for sale under the tradename LSFG-Retflow.

The non-invasive ocular blood flow sensor system can include a laser Doppler flowmeter (LDF), such as a confocal scanning laser Doppler flowmetry (CSLDF) system. In an example, the LDF system can be configured with an energy source capable of generating EM energy at a frequency of about 384 THz (corresponding to a wavelength of about 780 nm). In an example, the CSLDF system can include a system from Heidelberg Engineering GmbH (Heidelberg, Germany) offered for sale under the tradename Heidelberg Retina Flowmeter.

The non-invasive ocular blood flow sensor system can include an ocular coherence tomography angiography (OCTA) system. In an example, the function of an ocular coherence tomography (OCT) system can be enhanced, such as by placing an OCTA module in communication with the OCT system. An OCTA module can include control circuitry that can execute coded instructions to cause the OCT system to repeatedly scan a section of eye tissue, store each scan of eye tissue into memory, and process the stored scans to identify differences between scans, such as to generate an indication of ocular blood flow. In an example, the OCTA system can include at least one of an OCT system from Heidelberg Engineering GmbH (Heidelberg, Germany) offered for sale under the tradename Spectralis or an OCTA module from Heidelberg Engineering GmbH (Heidelberg, Germany) offered for sale under the tradename Spectralis OCT Angiography Module.

The non-invasive ocular blood flow sensor system can include a laser doppler velocimetry (LDV) system. In an example, the LDV system can be configured with an energy source capable of generating EM energy at a frequency of about 444 THz (corresponding to a wavelength of about 675 nm).

The non-invasive ocular blood flow sensor system can include a retinal vessel analyzer (RVA) system. The RVA system can include a system that illuminates the eye vessel and senses at least one of a coefficient of light reflection or a coefficient of light absorption.

The non-invasive ocular blood flow sensor system can include a doppler optical coherence tomography (DOCT) system with a collimated light source, such as a collimated light source configured to illuminate ocular tissue and a CCD sensor configured to receive the collimated light reflected from the ocular tissue. In an example, the DOCT system can be configured with an energy source capable of generating EM energy at a frequency of about 356 THz (corresponding to a wavelength of about 841 nm). In an example, the DOCT system can include the DOCT system from Optovue, Inc (Fremont, Calif.) offered for sale under the tradename RTVue.

The non-invasive ocular blood flow sensor system can include at least one of a retinal functional imager (RFI) system, a pulsatile ocular blood flow (POBF) system, a fundus pulsation amplitude (FPA) system, a fluorescein and Indocyanine Angiography (FA, ICG) system, a color doppler imaging (CDI) system, a retinal oximetry system, a magnetic resonance imaging (MRI) system, a magnetic resonance imaging (MRI) system, a blue light entoptoscopy) system, a frequency domain optical coherence tomography (FD-OCT) system, an angiography system, or a Split Spectrum Amplitude Decorrelation Angiography with Optical Coherence Tomography (SSADA-OCT) system.

The non-invasive ocular imaging system can include an electroretinography (ERG) system, such as at least one of a full field, multifocal, pattern, or visual evoked potential (VEP) electroretinography system. In an example, the ERG system can be configured with an energy source capable of generating EM energy at a frequency of about 440 THz (corresponding to a wavelength of about 680 nm or greater). In an example, the ERG system can include a system from Diopsys, Inc. (Pine Brook, N.J.) offered for sale under the tradename Diopsys Nova-ERG.

The ERG system can include a recording electrode, such as to sense an indication of electrical activity in the eye, including at least one of a neural and a non-neuronal cell in the retina, from stimulus applied to the eye including EM energy such as visible light. In an example, the recording electrode can be used with an ERG system to measure an indication of electrical activity in the eye, such as a pattern electroretinography (PERG) test as an indication of ocular blood flow. The recording electrode can include at least one of an electrode that can be in contact with the eye, such as an electrode attached to a contact lens and configured for contact with a surface of the eye, or an electrode in proximity to the eye, such as an electrode that can be located on the lower eye lid of an eye.

The non-invasive ocular imaging system can include a retinal functional imaging (RFI) system. The RFI system can be configured with an energy source capable of generating EM energy at a frequency of about 547 THz (corresponding to a wavelength of about 548 nm). In an example, the RFI system can include a system from Optical Imaging, Ltd. (Rehovot, Israel) offered for sale under the tradename RFI 3000. The RFI system can be configured with an energy source capable of generating EM energy at a frequency of about 666 THz (corresponding to a wavelength of at least 450 nm). In an example, the RFI system can include a system from OcuScience Inc. (Ann Arbor, Mich.) offered for sale under the tradename OcuMet Beacon.

The sensor 130 can include a force sensor, such as to sense force applied to patient tissue. The force sensor can located on the cover 110, such as to sense applied force between the cover 110 and patient tissue, such as when the cover 110 is located over the patient eye. The force sensor can be positioned at a specified location on the cover 110, such as to sense force applied to patient tissue at the location, or distributed around the peripheral edge of the cover 110, such as to sense force applied to patient tissue at any location around the periphery of the cover 110. In an example, the force sensor can be positioned between at least one of the cover 110 and the seal 119 or the patient interface surface 119A and patient tissue.

The force sensor can be located on at least one of the anterior plate 193 or the posterior plate 196. In an example, the force sensor can be configured to sense applied force between at least one of the anterior plate 193 and patient tissue or the posterior plate 196 and patient tissue, such as when at least one of the anterior plate 193 is located in contact with at least a portion of the anterior patient tissue or the posterior plate 196 is located over at least a portion of the posterior patient tissue.

The sensor 130 can include a patient response sensor, such as to receive input from a patient using the apparatus 100. The patient response sensor can be in communication with the control circuitry 140, such as wired or wireless communication, to transfer data received from the patient to the control circuit 140 for at least one of data processing or data recording. Patient input can include an indication of patient perception, such as effectiveness of an applied therapeutic regimen to adjust an indication of an indication of a headache symptom. Patient input can include patient commands, such as with regards to operation of the apparatus 100. In an example, the patient response sensor can adjust a parameter of the apparatus 100, such as an environmental parameter to increase or decrease the parameter, in response to patient input.

The patient response sensor can include a device to collect patient input, such as at least one of a smart device running an app configured to receive patient input and in communication with the control circuitry 140 or a fob device, such as an electro/mechanical device in communication with the control circuitry 140. The patient response sensor can include one or more selection buttons, such as to collect patient input. In an example, the one or more selection buttons can allow a patient to communicate an indication of patient perception, such as in response to an applied therapeutic regimen, to the control circuitry 140.

The control circuitry 140 can facilitate and coordinate operation of the apparatus 100. The control circuitry 140 can be coupled to, such as in communication with, at least one of the cover 110, such as an ETD associated with the cover 110, the fluid regulator 120, the sensor 130, the pressure source 150, the fluid source 170, or an adjunct device 160.

The control circuitry 140 can include a data interface. The data interface can be configured to receive a signal, such as at least one of an indication of the eye environment sensed by the sensor 130. The control circuitry 140 can process the received signal, such as into a processed signal, and transmit the processed signal to one or more components of the apparatus 100.

The data interface can be configured to transmit a signal, such as at least one of a remote data storage device or other computing machine 1400 remote from the apparatus 100 for subsequent data processing and data analysis.

The control circuitry 140 can be in communication with the fluid regulator 120, such as to adjust the position of the regulator valve to control the working fluid composition. The control circuitry 140 can be in communication with the sensor 130, such as to receive and process an indication of the eye environment including sensed data from the sensor 130. The control circuitry 140 can be in communication with the pressure source 150, such as to generate a pressure source control signal to adjust at least one of working fluid pressure or working fluid flow in the apparatus 100.

The control circuitry 140 can provide a communication interface, such as to allow for a user to operate and interact with the apparatus 100. The communication interface can include a graphical user interface (or GUI), such as communicate information to the user including information on the apparatus 100 (e.g., readout of sensed indications, fault status, etc) or receive information from the user. Information received from the user can include at least one of information to manage basic functionality of the apparatus 100, such as cycling the power to the apparatus 100, or an indication of user preference, such as operational parameters including target levels to define therapeutic protocols and safety parameter such as maximum and minimum limits. In an example, the communication interface can receive a safety pressure level, such as at least one of a maximum or minimum pressure level in the cavity 112 selected by the user to prevent damage to the patient eye, adjusting the working fluid pressure delivered to the cavity 112, or setting a target pressure level in the cavity 112.

The control circuitry 140 can include a digital signal processing (DSP) circuit, such as to receive and record an indication including an indication of the eye environment sensed by the sensor 130, such as at least one of an environmental parameter or a physiological parameter. The indication of the eye environment can be monitored and recorded by the control circuitry 140 for a duration, such as for a period of seconds, minutes, hours, days, years, or for the lifetime of the patient.

The control circuitry 140 can include a processing unit, such as a programmable central processing unit (CPU). The CPU can be configured to execute instructions to implement methods of using the apparatus 100, such as to treat, inhibit, prevent, or adjust an indication of an indication of a headache symptom experienced by a patient. In an example, the CPU can be a component of a computing machine, such as a computing machine 1400.

The CPU can be configured as a control circuit, such as a feedback control circuit. The feedback control circuit can receive information, such as at least one of an indication sensed by the sensor 130, an indication of user preference from the communication interface, or an indication of a processed signal including a signal processed by the CPU. The CPU can process the received information, such as to form a control signal, such as including at least one of a pressure source control signal or a tensioner control signal.

In an example, the pressure source control signal can be based on an indication of cavity pressure, such as pressure in the cavity 112, to achieve a target pressure level in the cavity 112. The pressure feedback control circuit can receive an indication of working fluid pressure in the cavity 112, such as an indication of cavity pressure level sensed by the sensor 130 including a pressure sensor in communication with the cavity 112. The pressure feedback control circuit can process the received indication of pressure level to form a control signal, such as a control signal to adjust the pressure source 150 to achieve the target pressure level in the cavity 112.

The CPU can be configured as a pressure feedback control circuit, such as to generate a control signal for the pressure source 150 (e.g., a pressure source control signal) to adjust pressure level in the cavity 112. The pressure level in the cavity 112 can be adjusted toward a target cavity pressure level, such as a headache target cavity pressure level selected to adjust patient perception of pain due to a headache symptom, including relief of patient pain. The pressure source control signal can be based on an indication sensed by a sensor 130, such as the difference between an indication sensed by a sensor 130 and the target cavity pressure level.

The CPU can be configured as a blood flow pressure feedback control circuit, such as to generate a blood flow pressure feedback control signal to adjust pressure level in the cavity 110 to vary ocular blood flow based on an indication of ocular blood flow. The indication of ocular blood flow can be sensed with the sensor 130, such as a blood flow sensor. The pressure level in the cavity can be adjusted toward a blood flow target cavity pressure level, such as the blood flow target cavity pressure level to adjust patient perception of pain due to a headache symptom, including relief of patient pain. In an example, the blood flow target cavity pressure level can be selected to minimize patient perception of pain.

The CPU can be configured as an applied force pressure feedback control circuit, such as to generate an applied force pressure feedback control signal to adjust pressure level in the cavity 110 to vary force applied to patient tissue, such as based on an indication of applied force between the cover 110 and patient tissue. The indication of applied force can be sensed with the sensor 130, such as a force sensor. The pressure level in the cavity can be adjusted toward an applied force target cavity pressure level, such as the applied force target cavity pressure level to adjust patient perception of pain due to a headache symptom, including relief of patient pain due to the headache symptom. In an example, the applied force target cavity pressure level can be selected to minimize patient perception of pain.

The CPU can be configured as a tensioner feedback control circuit, such as to generate a tensioner control signal to adjust tension level in a harness or tether of the apparatus 100. In an example, the tensioner control signal can be based on an indication of applied force, such as between the anterior plate 193 and anterior patient tissue, such as with a force sensor located between the anterior plate and anterior patient tissue. In an example, the tensioner control signal can be based on an indication of applied force, such as between the posterior plate 193 and posterior patient tissue, such as with a force sensor located between the posterior plate and posterior patient tissue. The tension in the harness or tether can be adjusted toward an applied plate force target tensioner level, such as the tension level to adjust patient perception of pain due to a headache symptom, including relief of patient pain due to the headache symptom. In an example, the applied plate force target tensioner level can be selected to minimize patient perception of pain.

The control circuitry 140 can include sweep circuitry, such as function-specific circuitry integrated into the control circuitry 140 or instructions implemented on the CPU. The sweep circuitry can be configured to adjust non-ambient pressure applied to the cavity 112, such as by generating a control signal to adjust the pressure source 150 to apply a pressure level to the cavity 112, such as in a specified pattern. A pressure range can be defined by a first pressure level and a second pressure level, such as where the second pressure level is greater than the first pressure level.

The sweep circuitry can be configured to perform a sweep test, such as to sequentially vary non-ambient pressure level applied to the cavity 112 over a pressure range. Sequential variation of non-ambient pressure can include sequentially increasing non-ambient pressure level over the pressure range, such as from the first pressure level to the second pressure level. Sequential variation of non-ambient pressure can include sequentially decreasing non-ambient pressure level over the pressure range, such as from the second pressure level to the first pressure level. A patient, such as a patient experiencing a headache symptom, can perform the sweep test and respond to each sweep pressure, such as by logging a response through the patient input sensor. Patient response to the sweep test can identify one or more target cavity pressures, such as a target cavity pressure that can relieve a headache symptom in the patient.

The sweep circuitry can be configured to define a target cavity pressure level, such as to adjust patient perception of an indication of a headache symptom to treat, inhibit, or prevent the headache symptom. The target cavity pressure level, such as the headache target cavity pressure value, can include a pressure level in the cavity 112, such as selected to adjust the patient perception associated with the headache symptom, such as to minimize patient perception of pain associated with the headache symptom.

The apparatus 100 can operate to identify or define a target cavity pressure level, such as a target cavity pressure level to adjust patient perception of an indication of a headache symptom to treat, inhibit, or prevent the headache symptom. The apparatus 100 can include a sensor 130, such as the patient response sensor, configured to receive patient input, such as patient perception of pain associated with an indication of a headache symptom as the non-ambient pressure in the cavity 112 is varied in the pressure range. In an example, the target cavity pressure value can be defined as the non-ambient pressure level applied to the cavity 112 that can minimize patient perception of pain associated with the headache symptom.

Processing the received indication of pressure can include calculating an indication, such as calculating an indication of the difference between the indication of cavity pressure level and an indication of user preference, including a cavity pressure setpoint level received from the communication interface to form an indication of a cavity pressure difference value. Processing the received indication can include generating a control signal based on the indication of cavity pressure difference value with a proportional-integral-derivative (PID) control algorithm running on the CPU to adjust the pressure source 150. Generating a control signal can include generating a control signal to minimize the difference between the received indication of pressure level and the cavity pressure setpoint level.

The CPU can be configured as a concentration feedback control circuit, such as to generate a regulator control signal to adjust a chemical constituent level in the cavity 112.

In an example, the regulator control signal can be based on an indication of a chemical constituent associated with the working fluid, such as an indication of nitric oxide (NO) concentration, to achieve a target NO concentration level in the working fluid. The concentration feedback control circuit can receive an indication of NO concentration level in the working fluid, such as an indication of NO level sensed by the senor 130 including a concentration sensor configured to sense NO.

The concentration feedback control circuit can process the received indication of NO level to form a control signal, such as a control signal to adjust the regulator 120 to achieve the target NO concentration level in the cavity 112.

Processing the received indication of NO concentration can include calculating the difference between the indication of NO concentration and an indication of user preference, including a NO setpoint level received from the communication interface, to form a NO difference value. Processing the received indication can include generating a control signal based on the NO difference value. Processing the received indication can include generating a control signal based on the NO difference value with a proportional-integral-derivative (PID) control algorithm running on the CPU to adjust the regulator 120. Generating a control signal can include generating a control signal to minimize the difference between the received indication of NO concentration and the NO setpoint level.

The control circuitry 140 can include a power source 152, such as to supply electrical energy to the apparatus 100. In an example, the power source 152 can include a battery, such as a lithium ion battery, and a transformer, such as to receive power from a wall outlet for use in the apparatus 100 at a specified voltage and current. The control circuitry 140 can include a heating element, such as a heating element in communication with the therapeutic fluid including a heating element located on a surface of or in proximity to the cover 110 including an inner surface 188 of the cover 110, or the fluid regulator 120, to increase the temperature of the therapeutic fluid.

The pressure source 150 can generate a volumetric flow of working fluid in the apparatus 100, such as to move working fluid from the pressure source 150 to the cavity 112 or to move working fluid from the cavity 112 to at least one of the pressure source 150 or to the surrounding environment. The pressure source 150 can be configured to apply non-ambient pressure to the cavity 112, such as to adjust an indication of fluid pressure including an indication of pressure level in the cavity 112, from a first pressure level to a second pressure level different from the first pressure level. A non-ambient pressure can include a pressure in the cavity 112 different from an ambient pressure, such as an ambient pressure surrounding the apparatus 100. In an example, a non-ambient pressure can include at least one of a positive non-ambient pressure, such as where the cavity pressure can be greater than the surrounding atmosphere, or a negative non-ambient pressure, such as where the cavity pressure can be less than the surrounding atmosphere.

The pressure source 150 can include a pump, such as a pump that can generate at least one of a positive gauge pressure or a negative gauge pressure. The pressure source 150 can include an electrically-powered pressure source, such as a pump including a displacement pump or a centrifugal pump. For example, the pressure source 150 can include a diaphragm pump, such as a diaphragm vacuum pump. The pressure source 150 can include a manually-powered pressure source, such as a hand pump including a bellows-style pump. In an example, the pressure source 150 can be integrated into a component of the apparatus 100, such as the cover 110.

The pressure source 150 can include a source of pressure, such as a pressurized gas cylinder or a source of pressurized fluid separate from the apparatus 100 that can be used to adjust working fluid pressure in the cavity 112. The pressure source 150 can include a source of pressure used in combination with a supplementary device to adjust pressure in the cavity. In an example, the pressure source 150 can include a venturi-type pump, such as a venturi jet pump, in communication with the source of pressure to adjust fluid pressure in the cavity 112.

The pressure source 150 can be characterized by physical characteristics, such as a relationship between physical characteristics. A useful measure for comparing the performance of several sources of flow includes a volume-pressure characteristic, such as the relationship between the volume of working fluid flow from a source of flow and the pressure, such as static pressure, created due to the fluid flow. In an example, the pressure source 150 can be characterized by a volume-pressure characteristic, such as a p-Q chart.

The pressure source 150 can generate a pressure in the cavity 112, such as to adjust pressure in the cavity 112 to move towards or achieve a target cavity pressure in the cavity 112. The target cavity pressure can include the cavity pressure to adjust patient perception of an indication of a headache symptom, such as a headache target cavity pressure.

The adjunct device 160 can apply energy to stimulate patient tissue, such as in combination with the apparatus 100, such as to affect a patient headache symptom. In an example, the adjunct device 160 can apply energy at a level, such as a headache target energy level, in combination with the apparatus 100, such as to minimize perceived patient pain associated with a headache symptom. The adjunct device 160 can include a neuromodulation device, such as an electrical stimulation neuromodulation device.

An electrical neuromodulation stimulation device can include a transcutaneous electrical nerve stimulation (TENS) device. A TENS device can include a power supply, such as to generate electrical stimulation energy, and an electrode, such as to transmit energy from the power supply to a patient.

The TENS device can include a facial nerve stimulator, such as a device to stimulate a supraorbital (or trigeminal) nerve. The facial nerve stimulator can include a device from CEFALY-Technology (Liege, Belgium) offered for sale under at least one of the tradenames Cefaly ACUTE, Cefaly DUAL, or Cefaly PREVENT.

The TENS device can include a cranial nerve stimulator, such as a device to stimulate at least one of a vagus nerve or a nerve originating at the trigeminal nerve nuclei. The vagus nerve stimulator can include a device from electroCore (Basking Ridge, N.J.) offered for sale under at least one of the trademarks GAMMACORE®, GAMMACORE SAPPHIRE®, or GAMMACORE VET®.

The TENS device can include a peripheral nerve stimulator, such as a device to stimulate at least one of a peripheral or a non-cranial nerve in the patient. The peripheral nerve stimulator can include a device from Theranica Bio-Electronics Ltd. (Netanya, Israel) offered for sale under the trademark NERIVIO MIGRA®.

An electrical neuromodulation stimulation device can include an implantable electrode device. An implantable electrode device can include a power supply, such as to generate electrical stimulation energy, and an implantable electrode, such as to transmit energy from the power supply to a patient.

The implantable electrode device can include an occipital nerve stimulator, such as a device to stimulate an occipital nerve in the patient. The implantable electrode device can include a spinal cord stimulator, such as a device to stimulate a spinal nerve including a dorsal column in the patient. The implantable electrode device can include a sphenopalatine ganglion stimulator, such as a device to stimulate the sphenopalatine ganglion. The implantable electrode device can include a deep brain stimulator, such as a device to stimulate the ventral tegmental area.

An electrical neuromodulation stimulation device can include a magnetic stimulation device. A magnetic stimulation device can include a power supply, such as to generate electrical stimulation energy, and a coil, such as to transmit energy from the power supply to a patient in the form of a magnetic field.

The magnetic stimulation device can include a transcranial magnetic stimulator (TMS) including a repetitive transcranial magnetic stimulator (rTMS), such as a device to stimulate at least a portion of the patient brain to affect a patient headache symptom. The magnetic stimulation device can include a single-pulse TMS (sTMS), such as a device to stimulate at least a portion of the patient brain to affect a patient headache symptom with a single pulse of magnetic energy. The sTMS can include a device from eNeura (Baltimore, Md.) offered for sale under the trademark sTMS Mini™.

The adjunct device 160 can include a pharmacological (or pharma) drug delivery device, such as for localized or systemic delivery of a drug to a patient to enhance the effect of therapies described elsewhere in this application including stimulation of patient tissue with the apparatus 100.

The pharma drug delivery device can include a timer or other notification device, such as to alert or remind a patient to administer a medication orally. In an example, acute headaches can be treated with the apparatus 100 adjusting applied non-ambient cavity pressure toward a target cavity pressure value and administration of a fast-acting headache relief drug, such as to adjust patient perception of the headache symptom to treat, inhibit, or prevent the headache symptom.

The pharma drug delivery device can include an infusion pump, such as to titrate a medication over an extended period of time. In an example, chronic headaches can be treated with the apparatus 100 adjusting applied non-ambient cavity pressure toward a target cavity pressure value and administration of a headache relief drug in the form of at least one of an orally-ingested, timed-release drug or titrated infusion with an infusion pump, such as to adjust patient perception of the headache symptom to treat, inhibit, or prevent the headache symptom.

A headache relief drug can include medications configured to adjust patient perception of an indication of a headache symptom. A headache relief drug can encompass many types of medications, such as antiepileptics, antidepressants, nerve blocks, non-steroidal analgesics, opioids, neuromodulators, and neurotoxic protein including botulinum toxin. A headache relief drug can include one or more classes of medications, such as general antagonists, triptans, and CRGP inhibitors.

A general antagonist can be used in the treatment of migraine-type headaches. A general antagonist can include any receptor antagonist that can be effective in inhibiting or preventing a headache, such as at least one of a NSAID (nonsteroidal anti-inflammatory drug) and a beta blocker. In an example a NSAID can include at least one of aspirin, ibuprofen, naproxen sodium, flurbiprofen, diclofenac potassium, indomethacin, or diclofenac potassium. In an example a beta blocker can include at least one of propranolol, timolol, atenolol, or nadolol.

A triptan can be used in the treatment of migraine-type and cluster-type headaches. A triptan can include a serotonin agonist, such as to cause constriction in blood vessels with 5-HT-1B and 5-HT-1D receptors. In an example, a triptan can include at least one of sumatriptan, rizatriptan, naratriptan, eletriptan, donitriptan, almotriptan, frovatriptan, avitriptan, or zolmitriptan.

A CRGP (calcitonin gene-related peptide) inhibitor can be used in the treatment of migraine-type headaches. A CRGP inhibitor can include at least one of a monoclonal antibody or a CGRP antagonist, such as to block the CGRP receptor of nerve cells. In an example, a CRGP inhibitor can include at least one of erenumab, fremanezumab, galcanezumab, eptinezumab, olcegepant, rimegepant, telcagepant, or ubrogepant.

The target cavity pressure can include the cavity pressure to affect a treatment of the patient eye, such as a cavity pressure prescribed by a medical professional to treat, inhibit, or prevent an eye condition. In an example, pressure in the cavity 112 can be adjusted with the pressure source 150 toward a target cavity pressure, such as a target cavity pressure to affect an indication of a physiological parameter of the patient eye including an indication of IOP level in the patient eye that can be sensed by a sensor 130 including a biosensor configured to sense an indication of IOP. Treatment of the patient eye can be affected by the pressure source 150, such as by adjusting the pressure source to achieve a target cavity pressure in the cavity 112 to affect a desired indication of IOP level in the patient eye.

The target cavity pressure can include a headache target cavity pressure, such as a pressure applied to the cavity 112 to adjust patient perception of an indication of a headache symptom, such as to treat, inhibit, or prevent the headache symptom. In an example, the headache target cavity pressure value can include a pressure level in the cavity 112 selected to minimize patient perception of pain associated with the headache symptom. The pressure level selected to minimize pain can be determined, such as by performing a headache sweep test with the apparatus 100 located on a patient. A headache target cavity pressure value can include cavity pressure level in a range of about 0 mmHg to about 100 mmHg, a cavity pressure level in a range of about 5 mmHg to about 40 mmHg, and a cavity pressure level in a range of about 10 mmHg to about 20 mmHg.

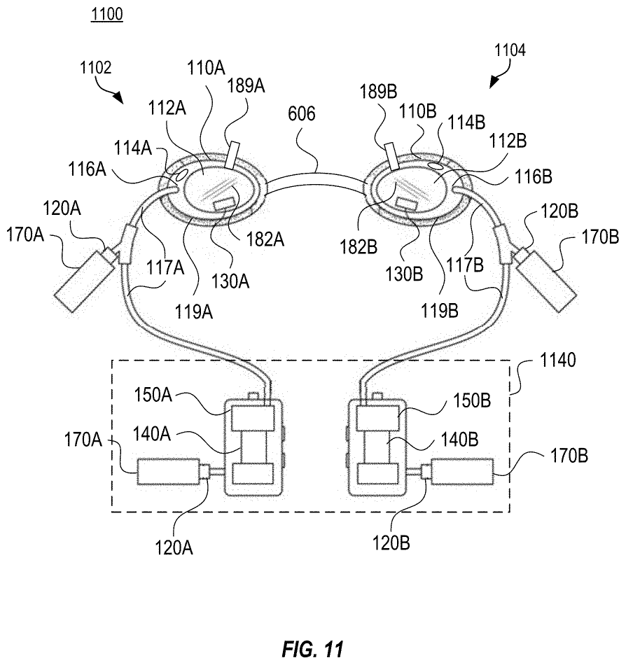

FIG. 11 shows an example of an apparatus 1100 that can control an eye environment over a patient eye, such as at least one of a left eye environment over the left patient eye or a right eye environment over the right patient eye. Controlling an eye environment can include at least one of establishing, adjusting, or maintaining an indication of the eye environment over the patient eye, such as an indication of working fluid cavity pressure in the cavity 112. In an example, control of the left eye environment can be independent of the right eye environment and control of the right eye environment can be independent of the left eye environment.

The apparatus 1100 can include a left system 1102 with a left cover 110A sized and shaped to fit over a left eye of a patient to define a left cavity 112A between the left cover 110A and an anterior surface of the left eye. The apparatus 1100 can include a right system 1104 with a right cover 110B sized and shaped to fit over the right eye of the patient to define a right cavity 112B between the right cover 110B and an anterior surface of the right eye. The apparatus 1100 can include a bridge 1106, such as to locate the left system 1102 with respect to the right system 1104. In an example, the left system 1102 can include at least one of the apparatus 100 and the right system 1104 can include at least one of the apparatus 100.

The apparatus 1100 can include system control circuitry 1140 to facilitate, coordinate, and control operation of the apparatus 1100. The system control circuitry 1140 can be configured to receive and process an indication of the eye environment, such as at least one of an indication of the left eye environment, an indication of the right eye environment, or an indication of a relationship between the indication of the left eye environment and the indication of the right eye environment.

The system control circuitry 1140 can include at least one of left control circuitry 140A, such as left control circuitry 140A to facilitate, coordinate, and control operation of the left system 1102, or right control circuitry 140B, such as right control circuitry 140B to facilitate, coordinate, and control operation of the right system 1102. In an example, the left control circuitry 140A can be configured to control operation of the left system 1102 independently of the right system 1104 and the right system 1104 can be configured to control operation of the right system 1104 independently of the left system 1102. In an example, the left control circuitry 140A can be capable of receiving and processing at least one of the indication of the left eye environment or the indication of the relationship between the left eye environment and the right eye environment. In an example, the right control circuitry 140B can be capable of receiving and processing at least one of the indication of the right eye environment or the indication of the relationship between the left eye environment and the right eye environment.

The system control circuitry 1140 can include pressure source circuitry, such as pressure source circuitry configured to adjust operation of the pressure source based on at least one of the indication of the left eye environment, the indication of the right eye environment, or the indication of a relationship between the indication of the left eye environment and the right eye environment. In an example, the pressure source circuitry can include at least one of left pressure source circuitry, such as coupled to the left control circuitry 140A, or right pressure source circuitry, such as coupled to the right control circuitry 140B.

The system control circuitry 1140 can be configured to facilitate, coordinate, and control operation of the apparatus 1100, such as in a master-slave control configuration. In an example, a first control circuitry can receive and process an indication of the eye environment and a second control circuitry, in communication with the first control circuitry, can receive the processed indication from the first control circuitry and adjust operation of the apparatus 1100, such as at least one of the left system 1102 or the right system 1104. In an example, the first control circuitry can include the left control circuitry 140A and the second control circuitry can include the right control circuitry 140B. In an example, the first control circuitry can include the right control circuitry 140B and the second control circuitry can include the left control circuitry 140A.

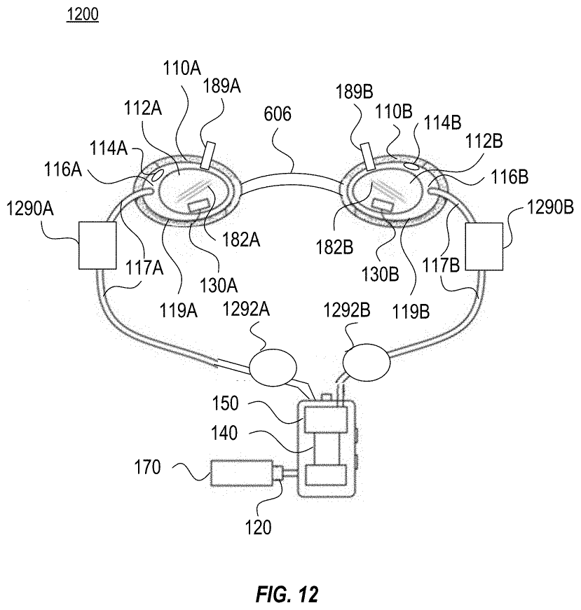

FIG. 12 shows an example of an apparatus 1200 that can control a left eye environment over a left eye of a patient and a right eye environment over a right eye of the patient, such as with a single pressure source. In an example, the apparatus 1200 can be operated with a single control system, such as the eye environment in the left cavity 112A and the eye environment in the right cavity 112B can be controlled to have the same eye environment in each cavity. For example, the apparatus 1200 can be operated, such as cavity pressure in the left cavity 112A can be equal to, such as approximately equal to, cavity pressure in the right cavity 112B.

The apparatus 1200 can include a cavity valve 1290, such as at least one of a left cavity valve 1290A or a right cavity valve 1290B. The apparatus 1200 can include a cavity reservoir 1292, such as at least one of a left cavity reservoir 1292A or a right cavity reservoir 1292B. In an example, the apparatus 1200 can be operated with independent control of the eye environment, such as control of the left eye environment can be independent of control of the right eye environment and control of the right eye environment can be independent of the left eye environment. For example, the apparatus 1200 can be operated, such as cavity pressure in the left cavity 112A can be different from cavity pressure in the right cavity 112B, such as by appropriate control of the cavity valve 1290 and the cavity reservoir 1292.

The apparatus 1200 can include a cavity valve 1290, such as the cavity valve 1290 in communication with the cavity 112. The cavity valve 1290 can be in communication with, such as coupled to, at least one of the sensor 130, the control circuitry 140, or the pressure source 150. In an example, the cavity valve 1290 can include at least one of a left control valve 1290A in communication with the left cavity 112A or a right control valve 1290B in communication with the right cavity 112B.

The cavity valve 1290 can control working fluid pressure in the cavity 112, such as to achieve a target cavity pressure in the cavity 112. Referencing FIG. 12, the left control valve 1290A can control working fluid pressure in the cavity 112A and the right control valve 1290B can control working fluid pressure in the cavity 112B.

The apparatus 1200 can include a cavity reservoir 1292, such as the cavity reservoir 1292 in communication with the cavity 112. The cavity reservoir 1292 can include at least one of a left cavity reservoir 1292A and a right cavity reservoir 1292B.

The cavity reservoir 1292 can serve to adjust an indication of system elastance in the apparatus 1200, such as to improve the ability of the apparatus 1200 to achieve a target cavity pressure. System elastance can be characterized by at least one of the ratio of change in pressure for a given change in volume, such as E=ΔP/ΔV, or the inverse of system compliance, such as C=1/E=ΔV/ΔP. In an example, an indication of system elastance can be equivalent to an indication of component elastance and an indication of system compliance can be equivalent to an indication of component compliance. A fluidic system with “high” elastance implies a fluidic system that can experience rapid pressure change as a function of volume change. In an example, an active cavity valve can fail to achieve the target cavity pressure in an apparatus 1200 with high elastance, such as due to slow feedback response resulting in overshooting the target cavity pressure. By adjusting elastance, such as by reducing system elastance or increasing system compliance, control of the apparatus 1200 can be improved, such as by reducing the rate of pressure change due to volume change to minimize feedback tracking error.

The cavity reservoir 1292 can include a supplementary volume, such as a volumetric space in communication with the cavity 112, including at least one of a fluidic accumulator or an expansion chamber. In an example, a supplementary volume can be defined as any additional volume of the cavity 112, such as any component in fluidic communication with the cavity 112, beyond the minimum volume required to convey pressure to the patient eye.

The amount of supplementary volume in the cavity reservoir 1292 can be selected, such as to adjust the system elastance to change system lag and error when pressurizing the apparatus 1200. Supplementary volume can be adjusted from a first supplementary volume level to a second supplementary volume. In an example, the second supplementary volume level can be less than the first supplementary volume level, such as to increase system elastance. In increasing system elastance, system lag for the apparatus 1200, including pressure system lag, can be reduced. In an example, the second supplementary volume level can be greater than the first supplementary volume level, such as to reduce system elastance. In reducing system elastance, system lag for the apparatus 800, including pressure system lag, can be increased.

The cavity reservoir 1292 can include a compliant portion of the apparatus 1200, such as a compliant portion of the apparatus 1200 in communication with the cavity 112. A compliant portion can include a portion of the apparatus 1200 in fluidic communication with the cavity 112, such as a portion of the apparatus 1200 that demonstrates a percentage variation in component compliance greater than the least compliant component of the cavity 112 or any component in fluidic communication with the cavity 112. The percentage variation in component compliance can be in a range of at least one of about 1% to about 25%, about 25% to about 50%, about 50% to about 75%, or about 75% to about 100% as compared to the least compliant component of the system.

The compliant portion can include an elastic portion, such as a portion of the apparatus 1200 in communication with the cavity 112 that demonstrates a percentage variation in component compliance greater than the least compliant component of the system. In an example, an elastic portion can include a membrane, such as the flexible septum as noted previously in this application.

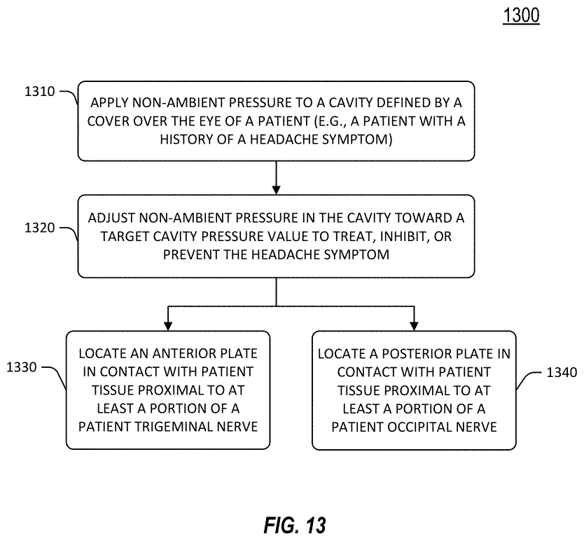

FIG. 13 shows an example method 1300 for using the apparatus 100 to adjust patient perception of an indication of a headache symptom. The control circuitry 140 can be configured to receive an indication from a user, such as an indication of a target cavity pressure level selected to minimize patient pain associated with a headache symptom, and process the received indication, such as to adjust the pressure level in the cavity 112 toward the indication of the target cavity pressure level received from the user. In an example, the control circuitry 140 can adjust the cavity pressure from a first indication of cavity pressure to a second indication of cavity pressure, such as approximately equivalent to the target cavity pressure level, such as to minimize patient pain associated with the headache symptom.

At step 1310, non-ambient pressure can be applied to a cavity 112, such as defined by a cover 110 over the eye of a patient, such as where the patient has a history of experiencing the headache symptom. Applying non-ambient pressure to the cavity 112 can include changing pressure in the cavity 112, such as from an ambient pressure to a non-ambient pressure.

Applying non-ambient pressure can include applying an external force to the cover 110, such as to compress at least one of the seal 119 or patient tissue, such as to decrease the volume of the cavity 112, such as to increase gauge pressure in the cavity 112. A positive pressure check valve, such as in communication with the cavity 112, can release positive gauge pressure from the cavity 112, such as limited by the cracking pressure of the positive pressure check valve. Applying non-ambient pressure can include releasing the external force from the cover 110, such as to allow the seal 119 or patient tissue to rebound, such as to decrease gauge pressure in the cavity 112. Applying non-ambient pressure can include regulating gauge pressure in the cavity 112, such as with a negative pressure check valve, such as with a cracking pressure selected to adjust non-ambient pressure in the cavity toward a target cavity pressure level.

Applying non-ambient pressure can include applying non-ambient pressure to the cavity 112 with a pressure source 150, such as toward a target cavity pressure level.

At step 1320, the non-ambient pressure applied to the cavity can be adjusted toward a target cavity pressure level, such as at least one of a headache target cavity pressure level or a force target cavity pressure level, to treat, inhibit, or prevent the headache symptom.

Adjusting the applied non-ambient pressure can include adjusting the cavity pressure toward an applied force target cavity pressure level. The applied force target cavity pressure level can be received, such as from a user specifying an applied force target cavity pressure level through an interface in communication with the control circuitry 140.

Adjusting the applied non-ambient pressure can include specifying a target cavity headache pressure value, such as by communicating the target cavity headache pressure value to the control circuitry 140 including inputting the value through a GUI attached to the control circuitry 140. Adjusting the applied non-ambient pressure can include operating a feedback control loop, such as running on the control circuitry 140, to minimize an error between the target cavity headache pressure value and an indication of cavity pressure in the cavity 112. In an example, cavity pressure can be sensed by a sensor 130, such as a pressure sensor located in communication with the cavity 112.

The applied force target cavity pressure level can be identified, such as by initiating a pressure sweep test with the sweep circuitry associated with the control circuitry 140 and collecting feedback from a user with a patient response sensor, such as from a user experiencing a headache symptom. The control circuitry 140 can process the data received, such as to identify one or more sweep pressure levels that can bring relief to the user and store the identified sweep pressure levels as one or more applied force target cavity pressure levels. The pressure sweep test can be conducted periodically, such as to update the applied force target cavity pressure levels.

At step 1330, an anterior plate 193 can be located in contact with patient tissue proximal to at least a portion of a patient trigeminal nerve. The apparatus 100 can include an anterior plate 193, such as attached to the cover 110. Locating the anterior plate in contact with patient tissue can include locating the cover 110 of the apparatus 100 over the patient eye so that the anterior plate 193 can contact the patient tissue. The anterior plate 193 can include an anterior plate harness 194.

Locating the anterior plate 193 can include tensioning the anterior plate harness 194, such as to generate plate contact pressure between the anterior plate 193 and the patient tissue. Tensioning the anterior plate can include specifying an applied force target tensioner level, such as by communicating the applied force target tensioner level to the control circuitry 140 including inputting the level through a GUI attached to the control circuitry 140. Adjusting the applied non-ambient pressure can include operating a feedback control loop, such as running on the control circuitry 140, to minimize an error between the applied force target tensioner level and an indication of tension in the anterior plate harness 194. In an example, tension in the anterior plate harness 194 can be sensed by a sensor 130, such as a tension sensor in communication with the anterior plate harness 194. In an example, force between the anterior plate 194 and the patient tissue can be sensed by a sensor 130, such as a force sensor located between the anterior plate 194 and the patient tissue.

Locating the anterior plate 193 can include adjusting non-ambient pressure in the cavity 112 toward a target cavity pressure, such as a non-ambient pressure in the cavity 112 adjusted toward a target cavity pressure to achieve a target plate contact pressure between the anterior plate 193 and the patient tissue proximal to the patient trigeminal nerve.

At step 1340, a posterior plate 196 can be located in contact with patient tissue proximal to at least a portion of a patient occipital nerve. Locating the posterior plate in contact with patient tissue can include locating the cover 110 of the apparatus 100 over the patient eye and adjusting the posterior plate tether 198 so that the posterior plate 196 can contact the patient tissue.

Locating the posterior plate 196 can include tensioning the posterior plate tether 198, such as to generate plate contact pressure between the posterior plate 196 and the patient tissue. Tensioning the posterior plate can include specifying an applied force target tensioner level, such as by communicating the applied force target tensioner level to the control circuitry 140 including inputting the level through a GUI attached to the control circuitry 140. Adjusting the applied non-ambient pressure can include operating a feedback control loop, such as running on the control circuitry 140, to minimize an error between the applied force target tensioner level and an indication of tension in the posterior plate tether 198. In an example, tension in the posterior plate tether 198 can be sensed by a sensor 130, such as a tension sensor in communication with the posterior plate tether 198. In an example, force between the posterior plate 196 and the patient tissue can be sensed by a sensor 130, such as a force sensor located between the posterior plate 196 and the patient tissue.

Locating the posterior plate 196 can include adjusting non-ambient pressure in the cavity 112 toward a target cavity pressure, such as a non-ambient pressure in the cavity 112 adjusted toward a target cavity pressure to achieve a target plate contact pressure between the posterior plate 196 and the patient tissue proximal to the patient occipital nerve.

A headache cover method can be formed from a sequence of steps, such as step 1310 and step 1320. The headache cover method can be implemented, such as by performing step 1310 and step 1320, such as to treat, inhibit, or prevent a headache symptom.

An anterior plate method can be formed from a sequence of steps, such as step 1310, step 1320, and step 1330. The anterior plate method can be implemented, such as by performing step 1310, step 1320, and step 1330, such as to treat, inhibit, or prevent a headache symptom.

A posterior plate method can be formed from a sequence of steps, such as step 1310, step 1320, and step 1340. The posterior plate method can be implemented, such as by performing step 1310, step 1320, and step 1340, such as to treat, inhibit, or prevent a headache symptom.

A combined plate method can be formed from a sequence of steps, such as step 1310, step 1320, step 1330, and step 1340. The combined plate method can be implemented, such as by performing a sequence of steps, such as at least one or more of step 1310, step 1320, step 1330, or step 1340 concurrently. In an example, a sequence of steps can include step 1310, step 1320, step 1330, and step 1340.

### Various Notes

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

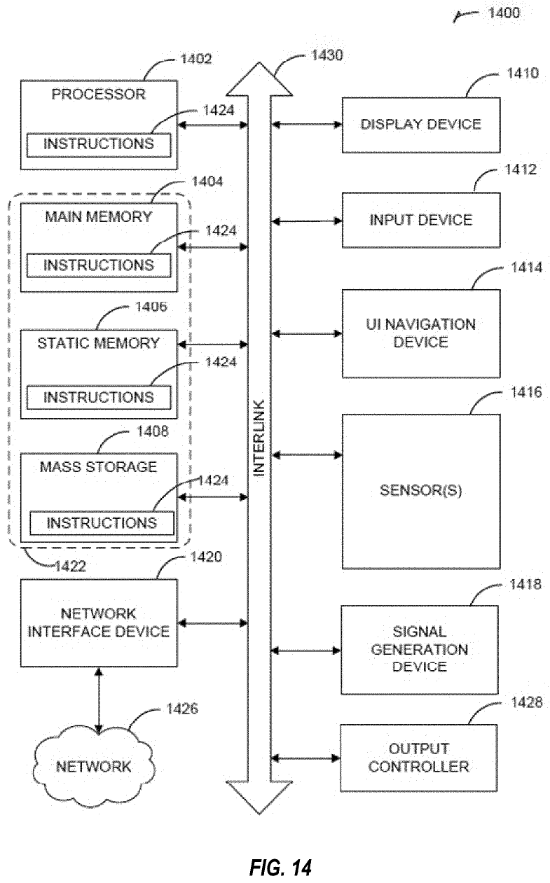

FIG. 14 shows an example block diagram of an example computing machine 1400 that can be used as control circuitry 140. Methods can be implemented on the control circuitry 140. The control circuitry 140 can include a computing machine 1400 upon which any one or more of the techniques or methods discussed herein can be performed. The machine 1400 may be a local or remote computer, or processing node in an on-the-go (OTG) device such as a smartphone, tablet, or wearable device. The machine 1400 may operate as a standalone device or may be connected (e.g., networked) to other machines. In an example, the machine may be directly coupled or be integrated with the apparatus 100, such as any components of the apparatus 100. It will be understood that when the processor 1402 is coupled directly to the apparatus 100, that some components of machine 1400 can be omitted to provide a lightweight and flexible device (e.g., display device, UI navigation device, etc.). In a networked deployment, the machine 1400 may operate in the capacity of a server machine, a client machine, or both in server-client network environments. In an example, the machine 1400 may act as a peer machine in peer-to-peer (P2P) (or other distributed) network environment. The machine 1400 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. In an example, the machine 1400 can include a purpose-designed circuit, such as a printed circuit board that can execute the functions and methods disclosed throughout this application. Further, while only a single machine is illustrated, the term “machine” can also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuitry can include a collection of circuits implemented in tangible entities that include hardware (e.g., simple circuits, gates, logic, etc.). Circuitry membership may be flexible over time and underlying hardware variability. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, the computer readable medium is communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time.

Machine (e.g., computer system) 1400 can include a hardware processor 1402 (e.g., a central processing unit (CPU), a graphics processing unit (GPU), a hardware processor core, or any combination thereof), a main memory 1404 and a static memory 1406, some or all of which may communicate with each other via an interlink (e.g., bus) 1408. The machine 1400 may further include a display unit 1410, an alphanumeric input device 1412 (e.g., a keyboard), and a user interface (UI) navigation device 1414 (e.g., a mouse). In an example, the display unit 1410, input device 1412 and UI navigation device 1414 may be a touch screen display. The machine 1400 may additionally include a storage device (e.g., drive unit) 1416, a signal generation device 1418 (e.g., a speaker), a network interface device 1420, and one or more sensors 1421, such as a global positioning system (GPS) sensor, compass, accelerometer, or other sensor. In an example, sensors 1421, such as including sensors 130, can include wearable, assistive device-based and environmental sensors, as described above. The machine 1400 may include an output controller 1428, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1416 may include a machine readable medium 1422 on which is stored one or more sets of data structures or instructions 1424 (e.g., software) embodying or utilized by any one or more of the techniques or functions described herein. The instructions 1424 may also reside, completely or at least partially, within the main memory 1404, within static memory 1406, or within the hardware processor 1402 during execution thereof by the machine 1400. In an example, one or any combination of the hardware processor 1402, the main memory 1404, the static memory 1406, or the storage device 1416 may constitute machine readable media.

While the machine readable medium 1422 is illustrated as a single medium, the term “machine readable medium” may include a single medium or multiple media (e.g., a centralized or distributed database, or associated caches and servers) configured to store the one or more instructions 1424.

The term “machine readable medium” may include any medium that is capable of storing, encoding, or carrying instructions for execution by the machine 1400 and that cause the machine 1400 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1424 may further be transmitted or received over a communications network 1426 using a transmission medium via the network interface device 1420 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1420 may include one or more physical jacks (e.g., Ethernet, coaxial, or phone jacks) or one or more antennas to connect to the communications network 1426. In an example, the network interface device 1420 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1400, and includes digital or analog communications signals or other intangible medium to facilitate communication of such software

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An apparatus to treat, inhibit, or prevent a headache symptom in a patient, the apparatus comprising:
a cover, sized and shaped to fit over a patient eye to define a cavity between the cover and the patient, the cover capable of applying and retaining non-ambient cavity pressure in contact with the patient to treat, inhibit, or prevent the headache symptom in the patient;
a pressure source, in communication with the cavity, capable of applying non-ambient cavity pressure to the cavity to treat, inhibit, or prevent the headache symptom in the patient;
a patient response sensor configured to receive a patient input including an indication of patient perception of an indication of the headache symptom; and
control circuitry, in communication with the pressure source, configured to control the pressure source to treat, inhibit, or prevent the headache symptom in the patient.

2. The apparatus of claim 1, in which the control circuitry is configured to control the pressure source to treat, inhibit, or prevent the headache symptom based at least in part on the patient input of the indication of the headache symptom.

3. The apparatus of claim 1, wherein the patient response sensor is configured to receive a patient input including an indication of patient perception of effectiveness of an applied therapeutic regimen to treat, inhibit, or prevent the headache symptom.

4. The apparatus of claim 1, in which the control circuitry is configured to control the pressure source to adjust the non-ambient pressure toward a headache target cavity pressure level to treat, inhibit, or prevent the headache symptom based at least in part on the patient input of the indication of the headache symptom.

5. The apparatus of claim 1, wherein the control circuitry includes sweep circuitry configured to vary non-ambient pressure level applied to the cavity in a specified pattern.

6. The apparatus of claim 5, wherein the sweep circuitry includes a central processing unit (CPU) executing an instruction set.

7. The apparatus of claim 5, wherein the specified pattern includes sequential variation of non-ambient pressure level applied to the cavity in a pressure range to identify a headache target cavity pressure level,
wherein the pressure range is defined by a first pressure level and a second pressure level-greater than the first pressure level.

8. The apparatus of claim 7, wherein the headache target cavity pressure level includes a non-ambient cavity pressure level in the pressure range selected to minimize patient perception of pain associated with the headache symptom.

9. The apparatus of claim 7, wherein the sweep circuitry is configured to sequentially vary non-ambient pressure from the first pressure level to the second pressure level.

10. The apparatus of claim 7, wherein the sweep circuitry is configured to sequentially vary non-ambient pressure from the second pressure level to the first pressure level.

11. The apparatus of claim 1, comprising an anterior plate attached to the cover and configured to contact patient tissue proximal to at least a portion of an anterior portion of the patient skull.

12. The apparatus of claim 11, comprising a force sensor, in communication with the control circuitry, to sense an indication of force applied by at least one of the cover or the anterior plate to patient tissue, and
wherein the control circuitry includes applied force pressure feedback control circuitry to adjust the cavity pressure toward the applied force target cavity pressure level.

13. The apparatus of claim 11, comprising a protuberance located on a patient interface surface between at least one of the cover or the anterior plate and the patient, wherein the protuberance is configured to apply an adjustable force to a patient acupressure point, and wherein the adjustable force is related to non-ambient cavity pressure in the cavity.

14. The apparatus of claim 1, comprising a posterior plate attached to the cover and configured to contact patient tissue proximal to at least a portion of a posterior portion of the patient skull.

15. The apparatus of claim 1, comprising an ocular blood flow sensor, in communication with the control circuitry, to sense an indication of ocular blood flow in the patient eye, and
wherein the control circuitry includes blood flow cavity pressure feedback control circuitry to adjust the cavity pressure toward the blood flow target cavity pressure level.

16. A method of using an apparatus to treat, inhibit, or prevent a headache symptom experienced by a patient, the apparatus including a cover, sized and shaped to fit over a patient eye to define a cavity between the cover and the patient, the cover capable of applying and retaining non-ambient cavity pressure in contact with the patient to treat, inhibit, or prevent the headache symptom in the patient, a pressure source, in communication with the cavity, capable of applying non-ambient cavity pressure to the cavity to treat, inhibit, or prevent the headache symptom in the patient, the method comprising:
forming the cavity over a patient eye; and
pressurizing the cavity to treat, inhibit or prevent the headache symptom.

17. The method of claim 16, wherein pressurizing includes adjusting non-ambient cavity pressure based at least in part on the patient input of the indication of the headache symptom.

18. The method of claim 16, wherein pressurizing includes adjusting non-ambient pressure toward a negative cavity pressure level relative to an environmental pressure outside of the cavity.

19. The method of claim 16, wherein pressurizing includes sequentially varying non-ambient pressure level applied to the cavity in a pressure range to identify a headache target cavity pressure level, wherein the pressure range is defined by a first pressure level and a second pressure level greater than the first pressure level.

20. A system to treat, inhibit, or prevent a headache symptom in a patient, the apparatus comprising:
a means to apply a non-ambient pressure over a patient eye to treat, inhibit, or prevent the headache symptom; and
a means to receive an indication of patient perception of an indication of the headache symptom to treat, inhibit, or prevent the headache symptom via the non-ambient pressure.
